(19) United States
(12) Patent Application Publication
(10) Pub. No.: US 2012/0195845 A1
(43) Pub. Date: Aug. 2, 2012

(54) DEODORANT AND DEODORIZING PRODUCT

(75) Inventor: Toshio Hirukawa, Nagoya-shi (JP)

(73) Assignee: TOAGOSEI CO., LTD., Tokyo (JP)

(21) Appl. No.: 13/446,317

(22) Filed: Apr. 13, 2012

Related U.S. Application Data
(62) Division of application No. 12/161,059, filed on Jul. 16, 2008.

(30) Foreign Application Priority Data
Feb. 3, 2006 (JP) 2006-027478
Feb. 28, 2006 (JP) 2006-052392

(51) Int. Cl. A61L 9/01

(52) U.S. Cl. 424/76.2

(57) ABSTRACT

An aldehyde gas deodorant dispersion composed of a mixture of an aminoguanidine salt that has a pH of 1 to 7 when dissolved in purified water and an acidic silica sol, the dispersion having a pH of 1 to 7, wherein a content of the aminoguannidine salt is 0.01 to 100 parts by weight relative to 100 parts by weight of a silica (SiO₂) content of the acidic silica sol. The deodorant dispersion can provide excellent deodorizing performance for an aldehyde gas, such as acetaldehyde or formaldehyde, and can further provide excellent deodorizing performance for various types of odors other than aldehyde gases. In addition, the deodorant dispersion can provide deodorizing performance over a wide range of temperatures.

The present application is a Divisional Application of U.S. application Ser. No. 12/161,059, filed on Jul. 16, 2008, which is a National Stage Application of PCT/JP2007/051578, filed on Jan. 31, 2007, which claims foreign priority to Japanese Application No. 2006-027478, filed on Feb. 3, 2006 and Japanese Application No. 2006-052392, filed Feb. 28, 2006, the entire contents of each of which are incorporated by reference herein.

### TECHNICAL FIELD

The present invention relates to a deodorant for aldehyde gases, and a deodorant composition comprising the deodorant and having excellent deodorizing performance for various types of bad odors other than aldehyde gases. Moreover, it relates to deodorizing products such as various types of fibers, paints, sheets, and moldings exhibiting excellent deodorizing performance as a result of addition of the deodorant or the deodorant composition.

### BACKGROUND ART

In recent years, consumer's needs for deodorization targeted at tobacco odor in particular have been increasing. Acetaldehyde is a main component of tobacco odor. Furthermore, health problems due to formaldehyde as seen in the sick house/sick building syndrome have been receiving attention. As removers for these aldehyde gases, it is mainly aldehyde removers comprising an amine compound that have been investigated.

Amine compounds have high affinity for aldehyde gases, and the possibility of removing an aldehyde gas in exhaust gas by contacting the exhaust gas containing the aldehyde gas with a solution of an amine compound is known (ref. e.g. Patent Publication 1). However, since liquid amine compounds have a strong and unpleasant odor, they are not suitable for application in daily life in a living space such as a living room or a kitchen.

Furthermore, a gas absorbent in which an amine compound is supported on an inorganic material is known, and this gas absorbent can withstand a thermal treatment when it is added to a resin, a paper, or a film.

For example, there are known gas absorbents in which an ammonium salt, aniline, etc. is supported on activated carbon (ref. e.g. Patent Publication 2 and Patent Publication 3), a compound having a primary amino group in the molecule is supported on a magnesium silicate-type clay mineral (ref. e.g. Patent Publication 4), or a polyamine compound is supported between layers of a layered phosphate (α-zirconium phosphate) (ref. e.g. Nonpatent Publication 1).

Furthermore, a hydrazine derivative having a solubility in water at 25° C. of 5 g/L or less (ref. e.g. Patent Publication 5), a deodorant comprising a magnesium silicate clay mineral and a compound having a primary amino group in the molecule (ref. e.g. Patent Publication 6), and a composition comprising a hydrazide compound in a synthetic resin (ref. e.g. Patent Publication 7) are known as aldehyde gas deodorants. However, these gas absorbents do not have a practical level of absorptive power for an aldehyde gas and, moreover, their aldehyde adsorbing performance is degraded by addition to a fiber or a paint.

Furthermore, it is known that deodorizing performance for acetaldehyde is exhibited by supporting an amino group-containing organosilicon compound on the surface of silica (ref. e.g. Patent Publication 8). With regard to this material, it becomes clear that, when used in combination with a deodorant having a high effect for a basic gas or a sulfurous gas, the intrinsic deodorizing performance that is expected to be exhibited cannot be exhibited sufficiently.

Moreover, there is a filter used for cleaning air that is formed by supporting an agent for removing aldehydes on a support such as activated carbon, and aminoguanidine sulfate is mentioned as the agent for removing aldehydes (ref. e.g. Patent Publication 9).

Furthermore, an aldehyde catcher comprising an aminoguanidine salt and water is known (ref. e.g. Patent Publication 10). It is known that the aminoguanidine salt has high aldehyde absorbability, and since it is water-soluble, it can be used in various types of products by dissolving it in water. However, the aminoguanidine salt not only exhibits no effect under some application conditions, but it also decomposes to show mutagenicity, and it is not disclosed or suggested that there are optimum application conditions.

An aldehyde deodorant composition comprising as effective components a silica sol and at least one type selected from the group consisting of an amino compound and a hydrazide compound has been reported (ref. e.g. Patent Publication 11).

In fuel cells employing as a fuel an alcohol such as methanol or ethanol, a filter comprising a nitrogen-containing compound such as ammonia, an amine compound, an amide compound, an imide compound, a urea-based compound, or a heterocyclic amine compound for absorbing a discharged intermediate product such as formaldehyde, formic acid, or methyl formate has been reported (ref. e.g. Patent Publication 12).

A patent application for an aldehyde gas deodorant comprising at least one type selected from the group consisting of a monoaminoguanidine salt, a diaminoguanidine salt, and a triaminoguanidine salt has been filed (ref. e.g. Patent Publication 13).

### PRIOR ART DOCUMENTS

(Patent Publication 1) JP-A-51-44587 (JP-A denotes a Japanese unexamined patent application publication)
(Patent Publication 2) JP-A-53-29292
(Patent Publication 3) JP-A-56-53744
(Patent Publication 4) JP-A-9-28778
(Patent Publication 5) JP-A-8-280781
(Patent Publication 6) JP-A-9-28778
(Patent Publication 7) JP-A-10-36681
(Patent Publication 8) JP-A-9-173830
(Patent Publication 9) JP-A-10-235129
(Patent Publication 10) JP-A-2005-97340
(Patent Publication 11) JP-A-2004-290543
(Patent Publication 12) JP-A-2006-261053
(Patent Publication 13) PCT/JP2005/19707
(Nonpatent Publication 1) Tsuhako et al., PHARM. TECH. JAPAN, 1996, Vol. 12, No. 12, p. 77-87

### DISCLOSURE OF INVENTION

### Problems to be Solved by the Invention

The present invention is to provide a deodorant and a dispersion having excellent deodorizing performance for an aldehyde gas such as acetaldehyde or formaldehyde and, furthermore, to provide a deodorant composition and a composition dispersion also having excellent deodorizing performance for various types of bad odors other than aldehyde gases. Moreover, it is to provide, using the deodorant etc., a deodorizing product such as a fiber, a paint, a sheet, or a molding exhibiting excellent deodorizing performance.

### Means for Solving the Problems

As a result of an intensive investigation by the present inventors, it has been found that an aldehyde gas deodorant comprising a mixture of an aminoguanidine salt that has a pH of 1 to 7 when dissolved in purified water and at least one type selected from a silicate compound that has a pH of 2 to 8 when dispersed in purified water, a tetravalent metal phosphate compound that has a pH of 2 to 8 when dispersed in purified water, a zeolite that has a pH of 2 to 8 when dispersed in purified water, and a silica gel that has a pH of 2 to 8 when dispersed in purified water, an aqueous suspension of the mixture having a pH of 1 to 7, has excellent performance as a deodorant for an aldehyde gas, and the present invention has thus been accomplished. Furthermore, the deodorant exhibits deodorizing performance over a wide range of temperatures. That is, the present invention is

(1) an aldehyde gas deodorant comprising a mixture of an aminoguanidine salt that has a pH of 1 to 7 when dissolved in purified water and at least one type selected from a silicate compound that has a pH of 2 to 8 when dispersed in purified water, a tetravalent metal phosphate compound that has a pH of 2 to 8 when dispersed in purified water, a zeolite that has a pH of 2 to 8 when dispersed in purified water, and a silica gel that has a pH of 2 to 8 when dispersed in purified water, an aqueous suspension of the mixture having a pH of 1 to 7,
(2) the aldehyde gas deodorant according to 1 above, wherein the temperature when carrying out the mixing is from room temperature to less than 60° C.,
(3) the aldehyde gas deodorant according to 1 or 2 above, wherein the mixture comprises an aminoguanidine salt that has a pH of 1 to 7 when dissolved in purified water, and a silicate compound that has a pH of 2 to 8 when dispersed in purified water or a silica gel that has a pH of 2 to 8 when dispersed in purified water,
(4) a deodorant composition comprising the aldehyde gas deodorant according to any one of 1 to 3 above and at least one type of deodorant selected from a sulfurous gas deodorant, a basic gas deodorant, and an organic acidic gas deodorant,
(5) a deodorant composition for an acidic gas, comprising an organic acidic gas deodorant and the aldehyde gas deodorant according to any one of 1 to 3 above,
(6) an aldehyde gas deodorant dispersion or a deodorant composition dispersion in which water and a dispersant and/or a surfactant are added to the aldehyde gas deodorant according to any one of 1 to 3 above or the deodorant composition according to 4 above, the dispersion having a pH of 1 to 7,
(7) an aldehyde gas deodorizing dispersion comprising a mixture of an aminoguanidine salt that has a pH of 1 to 7 when dissolved in purified water and an acidic silica sol, the dispersion having a pH of 1 to 7,
(8) the aldehyde gas deodorant dispersion according to 6 above, wherein it further comprises a lower alcohol,
(9) an aldehyde gas deodorant dispersion, a deodorant composition dispersion, an aldehyde gas deodorizing dispersion, or a deodorizing composition dispersion comprising the aldehyde gas deodorant dispersion, the deodorant composition dispersion, or the aldehyde gas deodorizing dispersion according to any one of 6 to 8 above and, further, a humectant,
(10) the aldehyde gas deodorant dispersion, the deodorant composition dispersion, or the aldehyde gas deodorizing dispersion according to 9 above, wherein the humectant is a polyhydric alcohol-based compound or urea,
(11) a deodorizing product produced by using the aldehyde gas deodorant or the deodorant composition according to any one of 1 to 5 above,
(12) a deodorizing product produced by carrying out coating, spray-coating, or mixing using the aldehyde gas deodorant dispersion, the deodorant composition dispersion, or the aldehyde gas deodorizing dispersion according to any one of 6 to 11 above,
(13) the deodorizing product according to 11 or 12 above, wherein it is a deodorizing filter, and
(14) the deodorizing product according to 11 or 12 above, wherein it is a deodorizing polyurethane foam.

### Effects of the Invention

Since the aldehyde gas deodorant and dispersion of the present invention have excellent deodorizing performance for aldehyde gases, aldehyde gases can be removed efficiently from an enclosed space such as the interior of a room or vehicle. Furthermore, a deodorizing product such as a fiber, a paint, a sheet, or a molding produced using the aldehyde gas deodorant or dispersion of the present invention has excellent deodorizing performance, and can reduce aldehyde gases volatilizing from the deodorizing product. Furthermore, the deodorant composition of the present invention can also efficiently remove bad odors other than aldehyde gas odor. For example, it can be used for cleaning exhaust gas containing formaldehyde and formic acid.

### BEST MODE FOR CARRYING OUT THE INVENTION

The present invention is explained in detail below. % denotes wt %, and parts denotes parts by weight. pH measurement is carried out at room temperature (1° C. to 30° C.), and preferably at 20° C. to 25° C. pH measurement of an aqueous suspension refers to a value when the pH of a supernatant of the aqueous suspension is measured.

### Aldehyde Gas Deodorant

The aldehyde gas deodorant of the present invention comprises a mixture of an aminoguanidine salt and at least one type selected from a silicate compound that has a pH of 2 to 8 when dispersed at 5% in purified water, a tetravalent metal phosphate compound that has a pH of 2 to 8 when dispersed at 5% in purified water, a zeolite that has a pH of 2 to 8 when dispersed at 5% in purified water, and a silica gel that has a pH of 2 to 8 when dispersed at 5% in purified water, an aqueous suspension of the mixture having a pH of 1 to 7. This aminoguanidine salt has a pH of 1 to 7 when dissolved in purified water. This pH measurement is carried out at room temperature.

With regard to the aldehyde gas deodorant of the present invention, the pH of a supernatant when dispersed at 5% in purified water at room temperature is no greater than 7, preferably no greater than 6.0, and more preferably no greater than 5.7, and the pH is at least 1, and preferably at least 1.5. It is preferable for the pH to be in this range since the aldehyde gas deodorant of the present invention then has high deodorizing performance for an aldehyde. Specifically, it is preferable since a large amount of an aldehyde gas can be deodorized, and the deodorization rate is high.

### Deodorant Composition

The deodorant composition of the present invention comprises an aldehyde gas deodorant and at least one type of deodorant selected from a sulfurous gas deodorant, a basic gas deodorant, and an organic acidic gas deodorant.

### Aldehyde Gas Deodorant Dispersion

The aldehyde gas deodorant dispersion of the present invention is a dispersion in which water and a dispersant and/or a surfactant are added to an aldehyde gas deodorant, the dispersion having a pH of 1 to 7.

### Deodorant Composition Dispersion

The deodorant composition dispersion of the present invention is a dispersion in which water and a dispersant and/or a surfactant are added to a deodorant composition, the dispersion having a pH of 1 to 7.

### Aldehyde Gas Deodorizing Dispersion

The aldehyde gas deodorizing dispersion of the present invention comprises at least an aminoguanidine salt and an acidic silica sol and has a pH of 1 to 7. With regard to the pH, the pH of a supernatant when dispersed at 5% is no greater than 7, preferably no greater than 6.0, and more preferably no greater than 5.7, and the pH is at least 1, and preferably at least 1.5. The aminoguanidine salt used here is preferably one having a pH of 1 to 7 when dispersed or dissolved in purified water.

### Aminoguanidine Salt

The aminoguanidine salt in the present invention may be any as long as a suspension of a mixture thereof with an inorganic powder has a pH of 1 to 7. Furthermore, a solution of the aminoguanidine salt, for example a 5% aqueous solution thereof, has a pH of 1 to 7, preferably a pH of 2 to 6, and yet more preferably a pH of 3.0 to 5.0. It may be used by preparing it to give this pH. It is preferable for the aminoguanidine salt to be in this range since a sufficient aldehyde deodorizing performance can be exhibited.

Examples of the aminoguanidine salt include aminoguanidine sulfate, aminoguanidine hydrochloride, diaminoguannidine hydrochloride, diaminoguanidine sulfate, and triaminoguanidine hydrochloride. They may be used singly or as a mixture in the present invention. As the aminoguanidine salt, aminoguanidine hydrochloride or aminoguanidine sulfate is particularly preferable from the viewpoint of safety.

Examples of the aldehyde gas that is a target for deodorization in the present invention include formaldehyde, acetaldehyde, propanal, butanal, and nonenal. However, that which is to be deodorized may contain an aldehyde gas and may be combined with another type of gas.

### Inorganic Powder

The inorganic powder in the present invention may be any as long as a suspension of a mixture thereof with the aminoguanidine salt has a pH of 1 to 7. The pH of a dispersion of the inorganic powder at 5% thereof is at least 2.0 but no greater than 8.0, preferably pH 3.0 to 7.5, and more preferably pH 4.0 to 7.0. It is preferable for the pH of a dispersion of the inorganic powder at 5 wt % to be in the above-mentioned range since the aldehyde deodorizing performance of the aminoguanidine salt can be exhibited even more and the mutagenicity is negative.

The inorganic powder in the present invention may be used without any limitation in components and shape as long as it can be mixed with the aminoguanidine salt and the pH is in the above-mentioned range, and it is preferably one that can improve the water resistance of the aldehyde gas deodorant of the present invention.

Examples of the inorganic powder include a silicate compound, a tetravalent metal phosphate compound, a zeolite, a silica gel, and an inorganic powder whose pH when dispersed at 5 wt % is adjusted to be in the above-mentioned range, and it is particularly preferable to use a silicate compound, a tetravalent metal phosphate compound, a silica gel, a pH adjusted mica, etc. since the deodorizing performance can be improved. Examples of the inorganic powder whose pH when dispersed at 5 wt % is adjusted to be in the above-mentioned range include mica, hydrotalcite, sepiolite, attapulgite, bentonite, and Y-type zeolite. An acid that is used for this pH adjustment is preferably an inorganic acid, and more preferably sulfuric acid or phosphoric acid.

### Silicate Compound

In the present invention, the silicate compound is preferably one having a pH of at least 2.0 but no greater than 8.0 when dispersed in purified water at 5 wt %, and more preferably one that can improve the water resistance of a mixture thereof with an aminoguanidine salt.

Specifically, aluminum silicate and magnesium silicate are preferable, amorphous aluminum silicate and amorphous magnesium silicate are more preferable from the viewpoint of the water resistance being improved, and amorphous aluminum silicate is yet more preferable since a mixture thereof with an aminoguanidine salt exhibits high aldehyde deodorizing performance under a high temperature atmosphere. They may be natural products or synthetic products. For example, a synthetic aluminum silicate is represented by Formula (1) below.

Al₂O₃.nSiO₂.mH₂O (1)

In Formula (1), n is a positive number of at least 6, preferably n is 6 to 50 and m is a positive number of 1 to 20, and particularly preferably n is 8 to 15 and m is 3 to 15.

Furthermore, a magnesium silicate is represented by Formula (2) below.

MgO.nSiO₂.mH₂O (2)

In Formula (2), n is a positive number of at least 1, preferably n is 1 to 20 and m is a positive number of 0.1 to 20, more preferably n is 1 to 15 and m is 0.3 to 10, and particularly preferably n is 3 to 15 and m is 1 to 8.

A synthetic silicate compound may be synthesized by, for example, the following means. It may be synthesized by mixing an aqueous solution of an aluminum salt or a magnesium salt and an aqueous solution of an alkali metal silicate, allowing it to stand under atmospheric pressure at room temperature, with the addition of an acid or an alkali as necessary, at a pH of about 3 to about 7, so as to form a coprecipitate, aging this at, for example, on the order of 40° C. to 100° C. or without aging, washing the coprecipitate with water, dehydrating, and drying.

The amount of water-soluble salt of aluminum and the amount of alkali metal silicate used in the synthesis of the aluminum silicate may be selected so that the SiO₂/Al₂O₃ molar ratio is at least 6, for example, in the range of 6 to 50, and preferably in the range of 8 to 15.

The amount of water-soluble salt of magnesium and the amount of alkali metal silicate used in the synthesis of the magnesium silicate may be selected so that the SiO₂/MgO molar ratio is at least 1, for example, in the range of 1 to 20, and preferably in the range of 1 to 15.

As another synthetic means, it may be formed by, for example, adding an aqueous solution of aluminum or magnesium salts to a silica sol, further mixing well uniformly while maintaining the pH of the system at about 3 to 7 with an acid or an alkali, further heating to, for example, on the order of 40° C. to 100° C., aging or not aging, then washing with water, dehydrating, and drying. In this process, the amount of silica sol and the amount of water-soluble salt of aluminum or magnesium may be selected in the same manner as for the above SiO₂/Al₂O₃ or SiO₂/MgO. In the above-mentioned explanation, examples in which the amorphous aluminum silicate and the amorphous magnesium silicate are synthesized individually are shown, but it is also possible to use a mixed aqueous solution of water-soluble salts of aluminum and magnesium so as to synthesize a compound containing both metals.

Examples of the water-soluble salt include water-soluble salts such as a sulfate, a nitrate, a chloride, an iodide, and a bromide.

Examples of the alkali and the acid used in the above-mentioned synthesis include an alkali such as sodium hydroxide, potassium hydroxide, sodium carbonate, potassium carbonate, or aqueous ammonia, and an acid such as hydrochloric acid, sulfuric acid, or nitric acid.

### Tetravalent Metal Phosphate Compound

In the present invention, the tetravalent metal phosphate compound is preferably a tetravalent metal phosphate compound that is insoluble in water or sparingly soluble in water and has a pH of at least 2.0 but no greater than 7.0 when dispersed in purified water at 5 wt %, and is more preferably one that can improve the water resistance of the aldehyde gas deodorant of the present invention.

Specific preferred examples thereof include zirconium phosphate, titanium phosphate, and tin phosphate. With regard to these compounds, there are amorphous compounds and crystalline compounds having various types of crystal systems such as α-type crystal, β-type crystal, γ-type crystal, and NASICON-type crystal, and any of these compounds may be used. Among them, it is preferable to use an α-type crystalline compound since it can improve the water resistance to a great extent, a mixture thereof with an aminoguanidine salt has high aldehyde deodorizing performance under a high temperature atmosphere, and it also has ammonia deodorizing properties.

### Silica Gel

In the present invention, the silica gel is preferably one having a pH of at least 2.0 but no greater than 7.0 when dispersed in purified water at 5 wt %, and more preferably one that can improve the water resistance of the aldehyde gas deodorant of the present invention.

Depending on the production process, silica gels having various properties are obtained by adjusting the surface area or pore size, and any known silica gel may be used as long the as the pH is in the above-mentioned range. As an example of the production, it is obtained by adding sulfuric acid to water glass, washing the gel thus obtained with water, drying, and then grinding.

### Zeolite

In the present invention, the zeolite is one having a pH of at least 2.0 but no greater than 8.0 when dispersed in purified water at 5 wt %, and preferably one having a pH of no greater than 7.0. Furthermore, it is more preferably one that can improve the water resistance of the aldehyde gas deodorant of the present invention.

The zeolite may be a natural product or a synthetic product. There are various types of structures for the zeolite, and any known zeolite may be used. Examples of the structures include A type, X type, Y type, α type, β type, and ZSM-5, and those having a pH other than a pH of 2 to 8 in a dispersion may be used by adjusting it to be in the range.

### Process for Producing Aldehyde Gas Deodorant of the Present Invention

A process for producing the aldehyde gas deodorant of the present invention is briefly explained.

With regard to the process for producing the aldehyde gas deodorant of the present invention, when producing a mixture from an inorganic powder or a dispersion thereof and an aminoguanidine salt or a solution or dispersion thereof, the mixture produced is one for which the pH of an aqueous suspension thereof is 1 to 7.

The aldehyde gas deodorant of the present invention may be produced by stirring an inorganic powder at a temperature from room temperature to less than 60° C., adding an aminoguanidine salt thereto, and mixing well. Alternatively, the aldehyde gas deodorant of the present invention may be produced by adding an aminoguanidine salt while stirring an inorganic powder at a temperature no greater than the decomposition temperature of the aminoguanidine salt, and mixing well. In these production processes, it is preferable to produce the aldehyde gas deodorant of the present invention at a temperature from room temperature to less than 60° C.

Moreover, the aldehyde gas deodorant of the present invention may be produced by stirring an inorganic powder at a temperature from room temperature to less than 60° C., adding thereto a solution of an aminoguanidine salt dropwise or by spraying, and mixing well. This mixture may be further dried. This drying is preferably carried out at 60° C. to 120° C., and more preferably 80° C. to 110° C., and may be carried out under reduced pressure. With regard to the treatment time of the drying process, the optimum time depends on the drying temperature, the amount to be treated, and the equipment, and it may be set according to the conditions.

Furthermore, the aldehyde gas deodorant of the present invention may be produced by stirring a dispersion of an inorganic powder at a temperature from room temperature to less than 60° C., adding an aminoguanidine salt thereto, and mixing well. This mixture may be further dried. This drying is preferably carried out at 60° C. to 120° C., and more preferably 80° C. to 110° C., and may be carried out under reduced pressure. With regard to the treatment time of the drying process, the optimum time depends on the drying temperature, the amount to be treated, and the equipment, and it may be set according to the conditions.

Moreover, the aldehyde gas deodorant of the present invention may be produced by stirring a dispersion of an inorganic powder at a temperature from room temperature to less than 60° C., adding thereto a solution or dispersion of an aminoguanidine salt, and mixing well. This mixture may be further dried. This drying is preferably carried out at 60° C. to 120° C., and more preferably 80° C. to 110° C., and may be carried out under reduced pressure. With regard to the treatment time of the drying process, the optimum time depends on the drying temperature, the amount to be treated, and the equipment, and it may be set according to the conditions.

In these production processes shown as examples, the methods for adding the inorganic powder and the aminoguanidine salt may be reversed. That is, the aldehyde gas deodorant of the present invention may be produced by stirring a solution or dispersion of an aminoguanidine salt at a temperature from room temperature to less than 60° C., adding thereto an inorganic powder, and mixing well. The same applies to production processes other than this production process.

Among these production processes, a production process employing a solution of an aminoguanidine salt is preferable.

The aldehyde gas deodorant of the present invention is preferably subjected to a heating treatment at 140° C. to 240° C. in order to further improve the water resistance, and more preferably a heating treatment at 160° C. to 220° C. The aldehyde gas deodorant of the present invention may be subjected to the drying step and the heating treatment in combination. With regard to the treatment time of the heating treatment, since the optimum time depends on the drying temperature, the amount to be treated, and the equipment, it may be set according to the conditions.

The solution of an aminoguanidine salt used in the present invention may be an aqueous solution or may employ an organic solvent such as an ethanol or methanol, and is preferably an aqueous solution. The dispersion of an inorganic powder may be an aqueous solution or may employ an organic solvent such as an alcohol or methanol, and is preferably an aqueous solution.

Furthermore, with regard to the aldehyde gas deodorant of the present invention, it is also possible to prepare a product containing the aldehyde gas deodorant of the present invention by adding dropwise, by spraying, etc. a solution of an aminoguanidine salt onto a filter, a fiber, a paper, etc. to which an inorganic powder has been adhered.

With regard to the proportions of the inorganic powder and the aminoguannidine salt in the aldehyde gas deodorant of the present invention, relative to 100 parts by weight of the inorganic powder, the aminoguanidine salt is 0.1 to 800 parts by weight, preferably 3 to 100 parts by weight, and more preferably 10 to 50 parts by weight. When the mixing ratio of the aminoguanidine salt is less than 0.1 parts by weight, a sufficient deodorizing effect cannot be obtained. Furthermore, it is not preferable for the mixing ratio of the aminoguanidine salt to exceed 800 parts by weight since the acetaldehyde deodorizing performance under an atmosphere of 80° C. might not be adequate, and it is not preferable since the aminoguanidine salt is not supported on the inorganic powder sufficiently and a deodorizing effect commensurate with the amount added cannot be expected.

Mixing with Another Deodorant

The aldehyde gas deodorant of the present invention is effective for an aldehyde gas, and examples of the aldehyde gas include acetaldehyde, formaldehyde, propanal, butanal, and nonenal. Furthermore, the aldehyde gas deodorant of the present invention may be used together with an aldehyde gas deodorant other than the aldehyde gas deodorant of the present invention. Examples of this aldehyde gas deodorant include ammonium sulfate, polyallylamine hydrochloride, EDTA sodium salt, triethanolamine, pyridine, dimethylhydantoin, casein, urea, thiourea, sodium caseinate, glycine, hexamethylenetetramine, guanidine nitrate, and hydroxylamine sulfate.

The application method of the aldehyde gas deodorant of the present invention may be targeted at an aldehyde gas on its own, but a deodorant other than one for an aldehyde gas may be mixed (deodorant composition), or may be used in combination. Furthermore, the aldehyde gas deodorant or the deodorant composition of the present invention can also improve the deodorizing properties by using it as a mixture with a magnesium silicate-type clay.

As a specific example of the deodorant that is mixed or used in combination with the aldehyde gas deodorant of the present invention, there is a basic gas deodorant for deodorizing a basic gas such as ammonia or trimethylamine. Examples of the basic gas deodorant include a tetravalent metal phosphate compound that is insoluble or sparingly soluble in water. Specific preferred examples of the tetravalent metal phosphate compound include zirconium phosphate, titanium phosphate, and tin phosphate. With regard to these compounds, there are amorphous compounds and crystalline compounds having various types of crystal systems such as α-type crystal, β-type crystal, γ-type crystal, and NASICON-type crystal, and any of these compounds may be mixed or used in combination with the aldehyde gas deodorant of the present invention as long as they have gas adsorptivity.

Furthermore, the aldehyde gas deodorant of the present invention may be mixed or used in combination with a sulfurous gas deodorant for deodorizing a sulfurous gas such as hydrogen sulfide or methylmercaptan. For example, the aldehyde gas deodorant of the present invention may be mixed or used in combination with zinc oxide, zinc silicate, or a tetravalent metal phosphate compound on which at least one type of metal ion selected from copper, zinc, and manganese is supported. Among the metal ions supported on the tetravalent metal phosphate compound, copper ion is particularly preferable since the deodorizing effect for hydrogen sulfide, etc. is high.

The supporting of metal ion on the tetravalent metal phosphate compound may be carried out by contacting the tetravalent metal phosphate compound with a solution of a salt of the metal ion, thus effecting ion exchange, etc.

The amount of metal ion supported may be freely adjusted as desired up to 100% as long as it is within the ion-exchange capacity of the tetravalent metal phosphate compound.

Furthermore, with regard to zinc oxide, copper silicate, and zinc silicate, those having a large specific surface area are preferable since the deodorizing performance is high.

Moreover, the aldehyde gas deodorant of the present invention may be mixed or used in combination with an organic acidic gas deodorant for deodorizing a bad odor such as acetic acid, isovaleric acid, or butyric acid. For example, a deodorant composition may be formed by mixing the aldehyde gas deodorant of the present invention with hydrated zirconium oxide or hydrated titanium oxide.

Hydrated zirconium oxide may be prepared by hydrolyzing a zirconium-containing solution such as an aqueous solution of zirconium oxychloride with water or an alkali solution. Hydrated zirconium oxide is known under various names such as zirconium oxyhydroxide, zirconium hydrooxide, hydrous zirconium oxide, and zirconium oxide hydrate, and all are the same as hydrated zirconium oxide.

A magnesium silicate type clay is a clay mineral containing magnesium silicate as a main component, and since it has pores with a pore size of about 1 nm it has gas adsorption capability. The aldehyde gas deodorant or the deodorant composition of the present invention to which a magnesium silicate type clay is added can further improve the deodorizing performance for a basic malodorous gas, an acidic malodorous gas, a sulfur-containing malodorous gas, and an aldehyde gas. Because of this, in the present invention, it is preferable to add a magnesium silicate type clay to the deodorant or the deodorant composition. In particular, adding a magnesium silicate type clay allows the deodorizing performance against nicotine, and pyridine, which is one of the main components of tobacco odor, to be improved.

Specific examples of the magnesium silicate type clay used in the present invention include sepiolite, silotile, loughlinite, and attapulgite.

It is preferable to add, relative to 100 parts by weight of the aldehyde gas deodorant of the present invention, 0.2 to 20 parts by weight of magnesium silicate type clay, and more preferably 0.5 to 10 parts by weight. When the magnesium silicate type clay is less than 0.2 parts by weight, improvement of the deodorizing performance might not be expected, and when it is added at greater than 20 parts by weight, improvement of the deodorizing performance might not be possible or the deodorizing performance against another malodorous gas might be degraded.

Either of the deodorant or the deodorant composition of the present invention described above is normally used in the form of a powder, and the average particle size thereof is preferably 0.01 to 50 μm, and more preferably 0.02 to 20 μm. It is not preferable for the average particle size to be less than 0.01 μm since handling is difficult, and there is the problem that it easily reaggregates. It is not very desirable for it to exceed 50 μm since, when it is dispersed in a surface treatment agent such as a binder and then applied to a fiber, etc. by postprocessing, it is difficult to disperse it uniformly in the surface treatment agent, and when it is added to a molding resin, a filter of a molding machine might be clogged, or dispersion might be poor, etc.

Furthermore, depending on the intended application, the aldehyde gas deodorant or the deodorant composition of the present invention may be granulated. In this case, each deodorant component may be granulated, or the deodorant composition may be granulated. With regard to a process for producing granules, any standard process that makes a powder into granules may be used. For example, there is a process in which granules are made by use of an alumina sol, a clay, etc. as a binder. The particle size may be adjusted to various values according to the hardness and density of the granules, pulverization strength, etc., but from the viewpoint of ease of handling it is preferably 0.1 to 3 mm.

The aldehyde gas deodorant composition of the present invention is a mixture of the aldehyde gas deodorant of the present invention with at least one type of material selected from a tetravalent metal phosphate compound, zinc silicate, a tetravalent metal phosphate compound on which at least one type of metal ion selected from copper, zinc, and manganese is supported, hydrated zirconium oxide, hydrated titanium oxide, zinc oxide, etc. The mixing proportions thereof are not particularly limited, and may be changed as appropriate depending on the environment in which the deodorant composition is used.

### Water Resistance

Water resistance may be imparted to the aldehyde gas deodorant of the present invention. The water resistance referred to here means that even after the deodorant is contacted with water once, there is little deterioration in the deodorizing performance. For example, after the deodorant is immersed in water once, when the deodorizing performance for acetaldehyde gas is measured, the percentage reduction compared with the deodorizing properties prior to the immersion is no greater than 65%, and preferably no greater than 50%. To explain test conditions in further detail, 1 g of the deodorant is placed in 100 mL of purified water at room temperature and stirred well, this suspension is filtered, further washed with 1000 mL of purified water, and dried at 110° C. The deodorant thus washed with water is subjected to measurement of deodorizing activity toward acetaldehyde gas, and the value is compared with the value prior to washing with water.

### High Temperature Deodorizing Properties

One of the characteristics of the aldehyde gas deodorant of the present invention is high deodorizing performance for an aldehyde gas under a high temperature atmosphere. The deodorizing performance under a high temperature atmosphere referred to here is the ability to suppress the generation of aldehyde gas when, for example, a fiber or resin molding containing the aldehyde gas deodorant of the present invention is heated. The deodorizing performance under a high temperature atmosphere being high referred to here means that it is possible to guarantee deodorizing properties under an environment of 40° C. to 90° C., and the concentration of aldehyde gas can be decreased down to a level that causes no problems. In other words, the deodorizing performance does not greatly deteriorate compared with the deodorizing performance at room temperature.

### Processing Method

The aldehyde gas deodorant of the present invention may be used as a deodorizing product by processing a powder, granules, or pellets thereof. For example, a deodorizing product may be made by packing a cartridge with the deodorant in the form of a powder, granules, or pellets. It is also possible to make a spray form of the deodorant using an aqueous solution of the aldehyde gas deodorant of the present invention or a suspension of a powder form of the deodorant. Alternatively, various types of deodorizing products may be formed by adding the aldehyde gas deodorant of the present invention to various types of product.

### Aldehyde Deodorant Dispersion

The aldehyde gas deodorant of the present invention may be dispersed in a dispersion medium, thus giving a deodorizing dispersion. Furthermore, the deodorizing dispersion may be prepared by dispersing an aminoguanidine salt and an inorganic powder. Production of the dispersion may employ any standard process for producing a dispersion of an inorganic powder. For example, production of the dispersion may be carried out by adding, to a dispersion medium such as water, an aminoguanidine salt, an inorganic powder and, as necessary a dispersant, a surfactant, an antifoaming agent, a humectant, a preservative, a viscosity adjusting agent, etc., and stirring by means of a sand mill, a disper, a ball mill, etc., thus carrying out dispersion.

Any dispersion medium may be used without limitation as long as it is water soluble and hydrophilic. Specific examples thereof include, as protic solvents, water and an alcohol. Examples of aprotic solvents include dimethylformamide, dimethylacetamide, dimethylsulfoxide, tetrahydrofuran, and acetone. They may be used singly or as a mixture of a plurality thereof. The dispersion medium is preferably water and/or an alcohol, and more preferably water.

As an alcohol as the dispersion medium, from the viewpoint of ease of handling, ethanol is preferable. When water and an alcohol are mixed and used as a dispersion medium, a preferred proportion of the alcohol added is 0.1 to 100 parts relative to 100 parts of water.

The dispersant used in the present invention is not particularly limited, and examples thereof include anionic surfactants such as an alkenyl succinate, an alkylbenzenesulfonate, an alkylnaphthalenesulfonate, an alkyl sulfate ester, a fatty alcohol sulfate ester, a polyoxyethylene alkyl ether sulfate ester, a dialkylsulfosuccinate, an alkyl phosphate ester, a phosphate ester-based copolymer, and a polycarboxylic acid type polymer surfactant, nonionic surfactants such as a polyoxyethylene alkyl ether, a polyoxyethylene alkyl aryl ether, and an organically modified organopolysiloxane, cationic surfactants such as an alkylamine salt and a quaternary ammonium salt, betaine-type amphoteric surfactants such as an alkylbetaine and an amidobetaine, a pyrophosphate, a tripolyphosphate, and a polyamine such as triethanolamine.

The dispersant is desirably a dispersant having an acidic functional group, and may have an interfacial activation effect. A plurality of the dispersants may be used, and there is no limitation as long as the aldehyde gas deodorant of the present invention can be dispersed in a dispersion medium. As the dispersant having an acidic functional group, a nonionic dispersant may be used.

The dispersant is preferably one comprising an acidic functional group-containing copolymer. Examples of the fundamental framework thereof include those comprising an ester chain, a vinyl chain, an acrylic chain, an ether chain, a urethane chain, etc., and some of the hydrogen atoms of these molecules may be replaced by a halogen atom. Among them, an acrylic resin, a polyester resin, and an alkyd resin are preferable, and an acrylic resin and a polyester resin are particularly preferable. Examples of the acidic functional group include a carboxyl group, a sulfonic group, and a phosphoric acid group, and among them a phosphoric acid group is preferable.

The acid value of the acidic functional group-containing dispersant in the present invention is preferably 5 to 150 mg KOH/g, and particularly preferably 30 to 130 mg KOH/g. It is not preferable for the acid value to be less than 5 mg KOH/g since the adsorptive power onto the surface of a deodorant particle might be insufficient, thereby degrading the dispersion stability. Furthermore, when the acid value exceeds 150 mg KOH/g, the proportion of a sterically repelling layer of the dispersant adsorbing on the surface of the deodorant particles becomes small, and sufficient dispersion stability of the deodorant particles might not be obtained. The acidic functional group may be completely randomly arranged in the resin molecule, but one in which the acidic functional group is positioned in a terminal portion of the molecule by a block or graft structure is preferable since it easily turns into a structure for stabilizing the dispersion of the deodorant particle due to solvation when the deodorant particle is adsorbed thereon. Examples of the counter cations include an alkali metal salt, an ammonium salt, and an amine salt, and an alkylammonium salt is particularly preferable.

A preferred weight-average molecular weight of the acidic functional group-containing dispersant is in the range of 800 to 100,000, and more preferably 800 to 10,000. It is not preferable for the molecular weight to be less than 800 since there are cases in which the dispersion effect is degraded, and it is not preferable for it to exceed 100,000 since aggregation or an increase in viscosity might occur.

The amount of dispersant added in the aldehyde gas deodorant dispersion of the present invention is, relative to 100 parts by weight of the inorganic powder, preferably 0.1 to 15 parts by weight, more preferably 0.5 to 12 parts by weight, and particularly preferably 1 to 10 parts by weight. It is not preferable for the amount of dispersant added to be less than 0.1 parts by weight since the dispersibility might not be sufficient and reaggregation might easily occur. Furthermore, it is not preferable for the amount of dispersant added to exceed 15 parts by weight since the dispersibility might be degraded due to the influence of excess dispersant, or the deodorizing properties might deteriorate.

Specific examples of the acidic functional group-containing dispersant include Disperbyk-110, Disperbyk-170, and Disperbyk-180 and -190 manufactured by BYK-Chemie, SER-AD FA192 manufactured by SERVODELDEN BV, Solsperse 3000, 9000, 13240, 13940, 17000, 17240, 17940, 21000, 24000, 26000, and 27000 manufactured by Zeneca Colors, Flowlen G-700 manufactured by Kyoeisha Chemical Co., Ltd., and Ajisper PA111 manufactured by Ajinomoto Co., Inc.

The antifoaming agent in the deodorant dispersion of the present invention may be any of a foam-breaking type, a foam-suppressing type, and a foam-removing type. Examples of the foam-breaking type include a polysiloxane solution.

The viscosity adjusting gent in the deodorant dispersion of the present invention may be of any type, and examples thereof include cellulose-based thickeners such as methylcellulose, carboxymethylcellulose, methylhydroxycellulose, methylhydroxypropylcellulose, and hydroxyethylcellulose, natural polysaccharides such as gum arabic, trangan gum, and guar gum, various types of polyacrylamide-based polymers, polyethylene oxide, and polyvinyl alcohol.

Adding a humectant to the deodorizing dispersion of the present invention (a separate description is given for the aldehyde gas deodorizing dispersion) enables the occurrence of nozzle orifice and strainer clogging to be suppressed when the deodorant dispersion or the deodorant composition dispersion, etc. is applied by a spray gun, etc., thereby stabilizing the liquid passage characteristics. The humectant that can be used is not particularly limited; examples thereof include polyhydric alcohol-based compounds such as polypropylene glycol, polyethylene glycol, xylitol, and d-sorbitol, and urea. It is preferable to use polyethylene glycol or urea since there is a high effect in suppressing the occurrence of nozzle orifice and strainer clogging, and polyethylene glycol is particularly preferable since the effect is high. There are various types of polyethylene glycols having different average molecular weights; one having an average molecular weight of 150 to 5000 is preferable since it has a high effect in suppressing clogging, and one having an average molecular weight of 194 to 1000 is more preferable. When the humectant is added, the proportion thereof added is, relative to 100 parts by weight of the solids content of the deodorant, preferably 0.01 to 10 parts by weight, and more preferably 0.1 to 5 parts by weight. It is not preferable for the proportion of the humectant added to be less than 0.01 parts by weight since the effect in suppressing clogging is not sufficient, and it is also not preferable for it to exceed 10 parts by weight since the effect in suppressing clogging does not increase in proportion to the amount added; instead, the drying properties of the spray-coated surface worsen, and the surface might become sticky after drying.

The solids content of the deodorant in the deodorant dispersion of the present invention is preferably 1 to 60 wt %, more preferably 3 to 40 wt %, and yet more preferably 5 to 25 wt %. It is not preferable for the deodorant solids content to be 1 wt % or less since the dispersion has a low viscosity and the dispersion stability might therefore become poor, and although it is possible to add an additive such as a viscosity adjusting agent in order to increase the viscosity of the dispersion, the additive might degrade the deodorizing performance. It is not preferable for the deodorant solids content to exceed 60 wt % since the viscosity of the dispersion becomes too high, the production thereof is therefore difficult, and the ease of handling of a product becomes poor.

It is also possible to add to the deodorant dispersion of the present invention a binder resin that is normally used for treating the surface of an acrylic acid-based or urethane-based fiber, a nonwoven fabric, a sheet, etc. In this case, the total of the binder resin and the deodorant solids content in the dispersion is preferably 5 to 50 wt % of the dispersion. With regard to the mixing ratio of the deodorant solids content and the binder resin in the dispersion, the binder resin solids content is preferably 10 to 300 parts by weight relative to 100 parts by weight of the deodorant solids content. It is not preferable for the binder resin solids content to be less than 10 parts by weight since, when the deodorizing dispersion is attached to a fiber, a nonwoven fabric, a sheet, etc., the adhesion is not sufficient, the deodorant comes off, and the deodorizing performance is degraded in some cases. Furthermore, it is not preferable for the binder resin solids content to exceed 300 parts by weight since, when it is processed into a fiber, a nonwoven fabric, a sheet, etc., the deodorant is covered with the resin, and the deodorizing performance is not sufficiently exhibited.

### Acidic Silica Sol

With regard to the acidic silica sol in the present invention, this solution is preferably acidic, more preferably has a pH of 2 to 6, and yet more preferably has a pH of 2 to 5. A process for producing the acidic silica sol involves, for example, preparing a sodium-stabilized silica sol using as a starting material sodium silicate, which is an alkali metal silicate, and then removing the alkali portion by an operation such as ion exchange. A dispersion medium for the acidic silica sol in the present invention is an aqueous solvent or an alcoholic solvent, and an aqueous solvent is preferable. The average particle size of the acidic silica sol is 3 to 250 nm, preferably 5 to 50 nm, and more preferably 8 to 30 nm. The average particle size referred to here is an average particle size of colloidal silica particles in the acidic silica sol.

Examples of commercial acidic aqueous silica sols include SNOWTEX O (product name, manufactured by Nissan Chemical Industries, Ltd.), SNOWTEX OS (product name, manufactured by Nissan Chemical Industries, Ltd.), SNOWTEX OXS (product name, manufactured by Nissan Chemical Industries, Ltd.), Nalco 1034A (product name, manufactured by Nalco Chemical Company), Nyacol 2034D1 (product name, manufactured by Eka Chemicals AB), Cataloid SN (product name, manufactured by Catalysts & Chemicals Ind. Co., Ltd.), and ADELITE AT-20Q (product name, manufactured by ADEKA Corporation).

### Process for Producing Aldehyde Gas Deodorizing Dispersion

A process for producing the aldehyde gas deodorizing dispersion of the present invention is briefly explained.

The aldehyde gas deodorizing dispersion of the present invention may be produced by stirring an acidic silica sol at a temperature from normal temperature to on the order of 60° C., adding thereto an aminoguanidine salt or a solution of an aminoguanidine salt, and stirring well.

It may be produced by reversing the order in which the silica sol and the aminoguanidine salt are added.

The solution of an aminoguanidine salt used here may employ an aqueous solution or an organic solvent such as an alcohol or methanol, and an aqueous solution is preferable.

It is particularly preferable to add a lower alcohol or a mixed liquid of water/lower alcohol to the aldehyde gas deodorizing dispersion of the present invention since the stability of the silica sol improves. Specific examples of the lower alcohol include methanol, ethanol, isopropyl alcohol, and ethylene glycol, and ethanol is particularly preferable since the effect in stabilizing the silica sol is high. The stability referred to here is expressed by a percentage change in the degree of cloudiness of the deodorizing dispersion. The degree of cloudiness may be evaluated by absorbance at 660 nm.

With regard to the proportions of the acidic silica sol and the aminoguannidine salt added in the aldehyde gas deodorizing dispersion of the present invention, relative to 100 parts by weight of the silica (SiO₂) content of the acidic silica sol, the aminoguanidine salt is 0.01 to 100 parts by weight, preferably 0.05 to 50 parts by weight, more preferably 0.1 to 30 parts by weight, and particularly preferably 0.1 to less than 10 parts by weight. When the proportion of the aminoguanidine salt is less than 0.01 parts by weight, a sufficient deodorizing effect cannot be obtained. Furthermore, it is not preferable for the proportion of the aminoguanidine salt to exceed 100 parts by weight since sufficient acetaldehyde deodorizing performance might not be obtained under an atmosphere of 80° C. That is, it is not preferable for the amount of aminoguanidine salt to be large since it is not adequately supported on the silica sol and a deodorizing effect that is commensurate with the amount of aminoguanidine salt added cannot be expected in some cases.

Adding a humectant to the aldehyde gas deodorizing dispersion of the present invention enables the occurrence of nozzle orifice and strainer clogging to be further suppressed when application is carried out by a spray gun, etc., thereby stabilizing the liquid passage characteristics. The humectant that can be used is not particularly limited; examples thereof include polyhydric alcohol-based compounds such as polypropylene glycol, polyethylene glycol, xylitol, and d-sorbitol, and it is preferable to use polyethylene glycol since there is a high effect in suppressing the occurrence of nozzle orifice and strainer clogging. There are various types of polyethylene glycols having different average molecular weights; one having an average molecular weight of 150 to 5000 is preferable since it has a high effect in suppressing clogging, and one having an average molecular weight of 194 to 1000 is more preferable. When the humectant is added, the proportion thereof added is, relative to 100 parts by weight of the solids content of the aldehyde gas deodorizing dispersion, preferably 0.01 to 10 parts by weight, and more preferably 0.1 to 5 parts by weight. It is not preferable for the proportion of the humectant added to be less than 0.01 parts by weight since the effect in suppressing clogging is not sufficient, and it is also not preferable for it to exceed 10 parts by weight since the suppressing effect does not increase in proportion to the amount added; instead, the drying properties of the spray-coated surface worsen, and the surface might become sticky after drying.

In the aldehyde gas deodorizing dispersion of the present invention, when it is an aqueous solution, a humectant may be used in combination. Examples of the humectant include water-soluble high molecular weight polymers such as polyvinyl alcohol, polyvinylpyrrolidone, polyethylene oxide, polypropylene oxide, and polyethylene glycol, and water soluble cellulose derivatives such as methylcellulose, hydroxyethylcellulose, and carboxycellulose; a water-soluble high molecular weight polymer is preferable, and one having a low molecular weight of no greater than on the order of 5000 is preferable. When the humectant is added, the proportion thereof added is, relative to 100 parts by weight of the deodorant, preferably 0.01 to 10 parts by weight, and more preferably 0.1 to 5 parts by weight.

Various types of surfactants may be added to the aldehyde gas deodorizing dispersion of the present invention as long as the aldehyde deodorizing performance is not impaired. In this case, a nonionic surfactant is preferable.

Supporting the aldehyde gas deodorizing dispersion of the present invention on a support such as a fiber, a nonwoven fabric, a wooden board, or a resin molding may be carried out by applying it to the surface of the support by coating or spraying or by immersing the support in the aldehyde gas deodorizing dispersion of the present invention. That is, the aldehyde gas deodorizing dispersion of the present invention may be supported on a support by coating, spraying, or immersing, and then drying. This drying may be carried out by natural drying or heating at a temperature of room temperature to on the order of 200° C. Furthermore, in order to strengthen the supporting on the support, a known binder resin such as an acrylic acid-based resin or a urethane-based resin may be used in combination with the aldehyde gas deodorizing dispersion of the present invention.

The aldehyde gas deodorizing dispersion, etc. of the present invention characteristically has high deodorizing performance for an aldehyde gas under a high temperature atmosphere. The deodorizing performance under a high temperature atmosphere referred to here means that, for example, it is possible to suppress the generation of an aldehyde gas when heating a fiber, a wooden board, a resin molding, etc. that has been treated with the aldehyde gas deodorizing dispersion, etc. of the present invention. The deodorizing performance under a high temperature atmosphere being high referred to here means that it is possible to guarantee deodorizing properties under an environment of 40° C. to 90° C., and the concentration of aldehyde gas can be decreased down to a level that causes no problems. In other words, the deodorizing performance does not greatly deteriorate compared with the deodorizing performance at room temperature.

### Application

The deodorants (the aldehyde gas deodorant, the aldehyde gas deodorant dispersion, and the aldehyde gas deodorizing dispersion) of the present invention have a deodorizing effect for aldehyde gases such as acetaldehyde, formaldehyde, and nonenal. Furthermore, the deodorant composition of the present invention has an excellent deodorizing effect for various types of bad odors of ammonia, hydrogen sulfide, methylmercaptan, etc. in addition to deodorization of aldehyde gases. From this, the deodorant or the deodorant composition of the present invention is effective in various fields where a conventional deodorant such as activated carbon is used, for example, in the fields of tobacco odor deodorization, domestic odor deodorization, body odor deodorization, excrement odor deodorization, garbage odor deodorization, etc.

The deodorant of the present invention may be used in a material in which aldehyde is generated from a substrate itself, for example, a construction material such as laminated wood, an assembled material, a flooring material, a particleboard, or an insulating material, a floor carpet, a sound deadening pad, a cushioning material, a car seat, a headrest, an armrest, a door trim, a molded ceiling, a sun visor, a rear package tray, an instrument panel, or a dash insulator, thus reducing aldehyde volatilizing from the substrate itself. Furthermore, the deodorant composition may be used in these applications.

Examples of deodorizing products comprising the deodorant of the present invention include deodorizing fiber, a deodorizing paint, a deodorizing sheet, and a deodorizing resin molding.

The deodorizing fiber comprising the deodorant of the present invention may be used in various fields where deodorizing properties are required. For example, the deodorizing fiber may be used in many fiber products including clothing, underwear, tights, socks, duvets, duvet covers, cushions, blankets, carpets, curtains, sofas, covers, seats, car seats, car mats, and air filters. With regard to a method for addition to a fiber product, there is a method involving attachment to the surface or the reverse face of a fiber product using a binder resin or a method in which it is kneaded into a fiber resin. Moreover, the deodorizing paint comprising the deodorant of the present invention may be utilized in various types of fields where deodorizing properties are required. For example, the deodorizing paint may be used for interior walls and exterior walls of buildings, interior walls of railway carriages, etc. Furthermore, the deodorizing sheet comprising the deodorant of the present invention may be utilized in various types of fields where deodorizing properties are required. For example, the deodorizing sheet may be used as medical packaging paper, food packaging paper, freshness-retaining paper, clothing made of paper, an air cleaner filter, wall paper, tissue paper, toilet paper, nonwoven fabric, paper, a filter, a film, etc. Furthermore, the deodorizing molding comprising the deodorant of the present invention may be utilized in various types of fields where deodorizing properties are required. For example, the deodorizing molding may be used in a household electrical appliance such as an air cleaner or a refrigerator, a general domestic utensil such as a garbage can or a drainer, various types of care equipment such as a portable toilet, or a daily product.

Furthermore, a filter prepared by adding the aldehyde gas deodorant or the deodorant composition of the present invention can prevent diffusion into air of formaldehyde, formic acid, etc. discharged while generating power or not generating power in a cell using as a fuel an alcohol such as methanol or ethanol or an ether such as dimethyl ether. Moreover, when the fuel cell is generating power, since water is discharged, the filter is required to have water resistance. Since the aldehyde gas deodorant, the deodorant composition, etc. of the present invention have excellent water resistance, they are suitable as a deodorant used in the filter. It is preferable to use one produced by using a deodorant composition in which the aldehyde gas deodorant of the present invention and an organic acidic gas deodorant are mixed.

### EMBODIMENTS

An aldehyde gas deodorant produced by uniformly mixing an inorganic powder and an aqueous solution of an aminoguanidine salt at a temperature of room temperature to less than 60° C.

An aldehyde gas deodorant produced by uniformly mixing an inorganic powder and an aqueous solution of an aminoguanidine salt at a temperature of room temperature to less than 60° C., and drying at 60° C. to 120° C.

An aldehyde gas deodorant produced by uniformly mixing an inorganic powder and a solution of an aminoguanidine salt at a temperature of room temperature to less than 60° C., drying at 60° C. to 120° C., and further heating at 140° C. to 240° C.

An aldehyde gas deodorant produced by uniformly mixing an inorganic powder and a solution of an aminoguanidine salt at a temperature of room temperature to less than 60° C., and heating at 140° C. to 240° C.

A process for producing the aldehyde gas deodorant according to any one of the above descriptions.

A process for producing an aldehyde gas deodorant involving producing a mixture by mixing an inorganic powder, an aminoguanidine salt, and water, wherein an aqueous suspension of the mixture has a pH of 1 to 7.

A process for producing an aldehyde gas deodorant involving producing a mixture by mixing a dispersion of an inorganic powder and a solution of an aminoguanidine salt, wherein an aqueous suspension of the mixture has a pH of 1 to 7.

A process for producing an aldehyde gas deodorant involving producing a mixture by mixing an inorganic powder and a solution of an aminoguanidine salt, wherein an aqueous suspension of the mixture has a pH of 1 to 7.

An aldehyde gas deodorant dispersion produced by mixing a dispersion of an inorganic powder and a solution of an aminoguanidine salt.

An aldehyde gas deodorant dispersion produced by mixing a dispersion of an inorganic powder and a solution of an aminoguanidine salt and adjusting the pH to 1 to 7.

An aldehyde gas deodorant dispersion produced by mixing well a dispersion of an inorganic powder and a solution of an aminoguanidine salt, and then adding a dispersant.

An aldehyde gas deodorant dispersion produced by mixing well a dispersion of an inorganic powder and a solution of an aminoguanidine salt with the pH adjusted to 1 to 7, and then adding a dispersant.

A process for producing the aldehyde gas deodorant dispersion according to any one of the above descriptions.

An aldehyde gas deodorizing dispersion wherein, with regard to an aldehyde gas deodorant comprising at least an acidic silica sol and an aminoguanidine salt, the proportion of the aminoguanidine salt is 0.01 to 100 parts by weight relative to 100 parts by weight of silica (SiO₂) content in the acidic silica sol.

An aldehyde gas deodorizing dispersion wherein, with regard to an aldehyde gas deodorant comprising at least an acidic silica sol and an aminoguanidine salt, the proportion of the aminoguanidine salt is 0.1 to less than 10 parts by weight relative to 100 parts by weight of silica (SiO₂) content in the acidic silica sol.

An aldehyde gas deodorizing dispersion prepared by adding an aminoguanidine salt to an acidic silica sol, and mixing at a temperature of room temperature to less than 60° C.

An aldehyde gas deodorizing dispersion prepared by adding an acidic silica sol to an aminoguanidine salt, and mixing at a temperature of room temperature to less than 60° C.

An aldehyde gas deodorizing dispersion prepared by adding an aqueous solution of an aminoguanidine salt to an acidic silica sol, and mixing at a temperature of room temperature to less than 60° C.

An aldehyde gas deodorizing dispersion prepared by adding an acidic silica sol to an aqueous solution of an aminoguanidine salt, and mixing at a temperature of room temperature to less than 60° C.

A deodorizing product produced using the aldehyde gas deodorant, the aldehyde gas deodorant dispersion, or the aldehyde gas deodorizing dispersion according to any one of the above descriptions.

A deodorizing particleboard produced using the aldehyde gas deodorant, the aldehyde gas deodorant dispersion, or the aldehyde gas deodorizing dispersion with a particleboard.

A deodorizing hardboard produced using the aldehyde gas deodorant, the aldehyde gas deodorant dispersion, or the aldehyde gas deodorizing dispersion with a hardboard.

A deodorizing kenaf board produced using the aldehyde gas deodorant, the aldehyde gas deodorant dispersion, or the aldehyde gas deodorizing dispersion with a kenaf board.

A deodorizing polyurethane foam produced using the aldehyde gas deodorant, the aldehyde gas deodorant dispersion, or the aldehyde gas deodorizing dispersion with a polyurethane foam.

A deodorizing felt produced using the aldehyde gas deodorant, the aldehyde gas deodorant dispersion, or the aldehyde gas deodorizing dispersion with a felt material such as cotton rag or polyester rag.

A deodorizing fabric cloth produced using the aldehyde gas deodorant, the aldehyde gas deodorant dispersion, or the aldehyde gas deodorizing dispersion with a cotton cloth, etc.

A deodorizing nonwoven fabric produced using the aldehyde gas deodorant, the aldehyde gas deodorant dispersion, or the aldehyde gas deodorizing dispersion with a nonwoven fabric, etc.

A deodorizing paper produced using the aldehyde gas deodorant, the aldehyde gas deodorant dispersion, or the aldehyde gas deodorizing dispersion with a paper, etc.

### EXAMPLES

The present invention is further explained specifically below, but is not limited to this explanation. Furthermore, % denotes wt %.

The method for preparing a deodorant composition sample, various evaluation test methods for the sample obtained, and results thereof are as described below.

### Aluminum Silicate

The aluminum silicate used in the examples was one having an SiO₂:Al₂O₃ molar ratio of 9:1 when synthesized, and the pH of a 5% suspension in purified water was 6.5.

### Example 1

While stirring 100 parts by weight of the aluminum silicate at room temperature, 50 parts by weight of a 30% aminoguanidine hydrochloride aqueous solution was added thereto. After the addition, stirring was carried out until uniform. Subsequently, after drying at 100° C. for 30 minutes, heating was carried out at 180° C. for 30 minutes, thus giving deodorant A. The pH of a 5 wt suspension of deodorant A in purified water was 5.5.

### Example 2

While stirring 100 parts by weight of the aluminum silicate at room temperature, 50 parts by weight of a 30% aminoguanidine sulfate aqueous solution was added thereto. After the addition, stirring was carried out until uniform. Subsequently, after drying at 100° C. for 30 minutes, heating was carried out at 210° C. for 30 minutes, thus giving deodorant B. The pH of a 5 wt suspension of deodorant B in purified water was 5.5.

### Example 3

While stirring 100 parts by weight of the aluminum silicate at room temperature, 50 parts by weight of a 30% diaminoguanidine hydrochloride aqueous solution was added thereto. After the addition, stirring was carried out until uniform. Subsequently, after drying at 100° C. for 30 minutes, heating was carried out at 180° C. for 30 minutes, thus giving deodorant C. The pH of a 5 wt suspension of deodorant C in purified water was 5.5.

### Example 4

While stirring 100 parts by weight of the aluminum silicate at room temperature, 50 parts by weight of a 30% triaminoguanidine hydrochloride aqueous solution was added thereto. After the addition, stirring was carried out until uniform. Subsequently, after drying at 100° C. for 30 minutes, heating was carried out at 180° C. for 30 minutes, thus giving deodorant D. The pH of a 5 wt suspension of deodorant D in purified water was 5.5.

### Example 5

The procedure of Example 1 was carried out in the same way except that α-zirconium phosphate (the pH of a 5% suspension thereof in water was 2.9, the same material being used below) was used instead of the aluminum silicate, thus giving deodorant E. The pH of a 5 wt % suspension of deodorant E in purified water was 2.2.

### Example 6

The procedure of Example 2 was carried out in the same way except that the α-zirconium phosphate was used instead of the aluminum silicate, thus giving deodorant F. The pH of a 5 wt % suspension of deodorant F in purified water was 2.2.

### Example 7

The procedure of Example 3 was carried out in the same way except that the α-zirconium phosphate was used instead of the aluminum silicate, thus giving deodorant G. The pH of a 5 wt % suspension of deodorant G in purified water was 2.2.

### Example 8

The procedure of Example 4 was carried out in the same way except that the α-zirconium phosphate was used instead of the aluminum silicate, thus giving deodorant H. The pH of a 5 wt % suspension of deodorant H in purified water was 2.2.

### Example 9

The procedure of Example 1 was carried out in the same way except that a silica gel (SYLYSIA 740 from Fuji Silysia Chemical Ltd., the pH of a 5 suspension thereof in water was 6.0, the same material being used below) was used instead of the aluminum silicate, thus giving deodorant I. The pH of a 5 wt suspension of deodorant I in purified water was 4.8.

### Example 10

The procedure of Example 2 was carried out in the same way except that the silica gel was used instead of the aluminum silicate, thus giving deodorant J. The pH of a 5 wt % suspension of deodorant J in purified water was 4.8.

### Example 11

The procedure of Example 3 was carried out in the same way except that the silica gel was used instead of the aluminum silicate, thus giving deodorant K. The pH of a 5 wt % suspension of deodorant K in purified water was 4.8.

### Example 12

The procedure of Example 4 was carried out in the same way except that the silica gel was used instead of the aluminum silicate, thus giving deodorant L. The pH of a 5 wt % suspension of deodorant L in purified water was 4.8.

### Example 13

The procedure of Example 1 was carried out in the same way except that ZSM5 zeolite (MIZUKASIEVES from Mizusawa Industrial Chemicals, Ltd., Si/Al=30, the pH of a 5% suspension thereof in water was 3.4) was used instead of the aluminum silicate, thus giving deodorant M. The pH of a 5 wt % suspension of deodorant M in purified water was 2.4.

### Example 14

The procedure of Example 2 was carried out in the same way except that the ZSM5 zeolite was used instead of the aluminum silicate, thus giving deodorant N. The pH of a 5 wt % suspension of deodorant N in purified water was 2.4.

### Example 15

The procedure of Example 3 was carried out in the same way except that the ZSM5 zeolite was used instead of the aluminum silicate, thus giving deodorant O. The pH of a 5 wt % suspension of deodorant O in purified water was 2.4.

### Example 16

The procedure of Example 4 was carried out in the same way except that the ZSM5 zeolite was used instead of the aluminum silicate, thus giving deodorant P. The pH of a 5 wt % suspension of deodorant P in purified water was 2.4.

Preparation of pH-Adjusted Mica

A pH-adjusted mica was prepared by adding 15 parts by weight of a 20% phosphoric acid aqueous solution to 100 parts by weight of SOMASIF ME-100 synthetic swelling mica (manufactured by Co-op Chemical Co., Ltd., the pH of a 5 suspension thereof in water was 10.7) while stirring at room temperature, and mixing well.

### Example 17

The procedure of Example 1 was carried out in the same way except that the pH-adjusted mica was used instead of the aluminum silicate, thus giving deodorant Q. The pH of a 5 wt % suspension of deodorant Q in purified water was 5.9.

### Example 18

The procedure of Example 2 was carried out in the same way except that the pH-adjusted mica was used instead of the aluminum silicate, thus giving deodorant R. The pH of a 5 wt % suspension of deodorant R in purified water was 5.9.

### Example 19

The procedure of Example 3 was carried out in the same way except that the pH-adjusted mica was used instead of the aluminum silicate, thus giving deodorant S. The pH of a 5 wt % suspension of deodorant S in purified water was 5.9.

### Example 20

The procedure of Example 4 was carried out in the same way except that the pH-adjusted mica was used instead of the aluminum silicate, thus giving deodorant T. The pH of a 5 wt % suspension of deodorant T in purified water was 5.9.

### Example 21

The procedure of Example 1 was carried out in the same way except that, after the aluminum silicate was stirred at 100° C. for 1 hour, the aminoguanidine hydrochloride solution was added thereto while stirring at 100° C., thus giving deodorant A (100). The pH of a 5 wt % suspension of deodorant A (100) in purified water was 5.5.

### Example 22

The procedure of Example 1 was carried out in the same way except that, after the aluminum silicate was stirred at 100° C. for 1 hour, the aminoguanidine sulfate solution was added thereto while stirring at 100° C., thus giving deodorant B (100). The pH of a 5 wt % suspension of deodorant B (100) in purified water was 5.5.

### Example 23

100 parts by weight of the aluminum silicate and 25 parts by weight of aminoguanidine hydrochloride were mixed well at room temperature until uniform, and then heated at 160° C. for 30 minutes, thus giving deodorant A1. The pH of a 5 wt % suspension of deodorant A1 in purified water was 4.3.

### Example 24

100 parts by weight of the aluminum silicate and 25 parts by weight of aminoguanidine sulfate were mixed well at room temperature until uniform, and then heated at 210° C. for 30 minutes, thus giving deodorant B1. The pH of a 5 wt suspension of deodorant B1 in purified water was 4.3.

### Example 25

100 parts by weight of the aluminum silicate and 25 parts by weight of diaminoguanidine hydrochloride were mixed well at room temperature until uniform, and then heated at 180° C. for 30 minutes, thus giving deodorant C1. The pH of a 5 wt % suspension of deodorant C1 in purified water was 4.3.

### Example 26

100 parts by weight of the aluminum silicate and 25 parts by weight of triaminoguanidine hydrochloride were mixed well at room temperature until uniform, and then heated at 180° C. for 30 minutes, thus giving deodorant D1. The pH of a 5 wt % suspension of deodorant D1 in purified water was 4.3.

### Example 27

The procedure of Example 23 was carried out in the same way except that the α-zirconium phosphate was used instead of the aluminum silicate, thus giving deodorant E1. The pH of a 5 wt % suspension of deodorant E1 in purified water was 1.7.

### Example 28

The procedure of Example 24 was carried out in the same way except that the α-zirconium phosphate was used instead of the aluminum silicate, thus giving deodorant F1. The pH of a 5 wt % suspension of deodorant F1 in purified water was 1.7.

### Example 29

The procedure of Example 25 was carried out in the same way except that the α-zirconium phosphate was used instead of the aluminum silicate, thus giving deodorant G1. The pH of a 5 wt % suspension of deodorant G1 in purified water was 1.7.

### Example 30

The procedure of Example 26 was carried out in the same way except that the α-zirconium phosphate was used instead of the aluminum silicate, thus giving deodorant H1. The pH of a 5 wt % suspension of deodorant H1 in purified water was 1.7.

### Example 31

The procedure of Example 23 was carried out in the same way except that the silica gel was used instead of the aluminum silicate, thus giving deodorant I1. The pH of a 5 wt % suspension of deodorant I1 in purified water was 4.1.

### Example 32

The procedure of Example 24 was carried out in the same way except that the silica gel was used instead of the aluminum silicate, thus giving deodorant J1. The pH of a 5 wt % suspension of deodorant J1 in purified water was 4.1.

### Example 33

The procedure of Example 25 was carried out in the same way except that the silica gel was used instead of the aluminum silicate, thus giving deodorant K1. The pH of a 5 wt % suspension of deodorant K1 in purified water was 4.1.

### Example 34

The procedure of Example 26 was carried out in the same way except that the silica gel was used instead of the aluminum silicate, thus giving deodorant L1. The pH of a 5 wt % suspension of deodorant L1 in purified water was 4.1.

### Example 35

The procedure of Example 23 was carried out in the same way except that the ZSM5 zeolite was used instead of the aluminum silicate, thus giving deodorant M1. The pH of a 5 wt % suspension of deodorant M1 in purified water was 1.9.

### Example 36

The procedure of Example 24 was carried out in the same way except that the ZSM5 zeolite was used instead of the aluminum silicate, thus giving deodorant N1. The pH of a 5 wt % suspension of deodorant N1 in purified water was 1.9.

### Example 37

The procedure of Example 25 was carried out in the same way except that the ZSM5 zeolite was used instead of the aluminum silicate, thus giving deodorant O1. The pH of a 5 wt % suspension of deodorant O1 in purified water was 1.9.

### Example 38

The procedure of Example 26 was carried out in the same way except that the ZSM5 zeolite was used instead of the aluminum silicate, thus giving deodorant P1. The pH of a 5 wt % suspension of deodorant P1 in purified water was 1.9.

### Example 39

The procedure of Example 23 was carried out in the same way except that the pH-adjusted mica was used instead of the aluminum silicate, thus giving deodorant Q1. The pH of a 5 wt % suspension of deodorant Q1 in purified water was 4.7.

### Example 40

The procedure of Example 24 was carried out in the same way except that the pH-adjusted mica was used instead of the aluminum silicate, thus giving deodorant R1. The pH of a 5 wt % suspension of deodorant R1 in purified water was 4.7.

### Example 41

The procedure of Example 25 was carried out in the same way except that the pH-adjusted mica was used instead of the aluminum silicate, thus giving deodorant S1. The pH of a 5 wt % suspension of deodorant S1 in purified water was 4.7.

### Example 42

The procedure of Example 26 was carried out in the same way except that the pH-adjusted mica was used instead of the aluminum silicate, thus giving deodorant T1. The pH of a 5 wt % suspension of deodorant T1 in purified water was 4.7.

### Example 43

100 parts by weight of the aluminum silicate and 25 parts by weight of aminoguanidine hydrochloride were mixed well at room temperature until uniform, thus giving deodorant A0. The pH of a 5 wt % suspension of deodorant A0 in purified water was 4.3.

### Example 44

100 parts by weight of the aluminum silicate and 25 parts by weight of aminoguanidine sulfate were mixed well at room temperature until uniform, thus giving deodorant B0. The pH of a 5 wt % suspension of deodorant B0 in purified water was 4.3.

### Example 45

100 parts by weight of the aluminum silicate and 25 parts by weight of diaminoguanidine hydrochloride were mixed well at room temperature until uniform, thus giving deodorant C0. The pH of a 5 wt % suspension of deodorant C0 in purified water was 4.3.

### Example 46

100 parts by weight of the aluminum silicate and 25 parts by weight of triaminoguanidine hydrochloride were mixed well at room temperature until uniform, thus giving deodorant D0. The pH of a 5 wt % suspension of deodorant D0 in purified water was 4.3.

### Example 47

The procedure of Example 43 was carried out in the same way except that the α-zirconium phosphate was used instead of the aluminum silicate, thus giving deodorant E0. The pH of a 5 wt % suspension of deodorant E0 in purified water was 1.7.

### Example 48

The procedure of Example 44 was carried out in the same way except that the α-zirconium phosphate was used instead of the aluminum silicate, thus giving deodorant F0. The pH of a 5 wt % suspension of deodorant F0 in purified water was 1.7.

### Example 49

The procedure of Example 45 was carried out in the same way except that the α-zirconium phosphate was used instead of the aluminum silicate, thus giving deodorant G0. The pH of a 5 wt % suspension of deodorant G0 in purified water was 1.7.

### Example 50

The procedure of Example 46 was carried out in the same way except that the α-zirconium phosphate was used instead of the aluminum silicate, thus giving deodorant H0. The pH of a 5 wt % suspension of deodorant H0 in purified water was 1.7.

### Example 51

The procedure of Example 43 was carried out in the same way except that the silica gel was used instead of the aluminum silicate, thus giving deodorant I0. The pH of a 5 wt % suspension of deodorant I0 in purified water was 4.1.

### Example 52

The procedure of Example 44 was carried out in the same way except that the silica gel was used instead of the aluminum silicate, thus giving deodorant J0. The pH of a 5 wt % suspension of deodorant J0 in purified water was 4.1.

### Example 53

The procedure of Example 45 was carried out in the same way except that the silica gel was used instead of the aluminum silicate, thus giving deodorant K0. The pH of a 5 wt % suspension of deodorant K0 in purified water was 4.1.

### Example 54

The procedure of Example 46 was carried out in the same way except that the silica gel was used instead of the aluminum silicate, thus giving deodorant L0. The pH of a 5 wt % suspension of deodorant L0 in purified water was 4.1.

### Example 55

The procedure of Example 43 was carried out in the same way except that the ZSM5 zeolite was used instead of the aluminum silicate, thus giving deodorant M0. The pH of a 5 wt % suspension of deodorant M0 in purified water was 1.9.

### Example 56

The procedure of Example 44 was carried out in the same way except that the ZSM5 zeolite was used instead of the aluminum silicate, thus giving deodorant N0. The pH of a 5 wt % suspension of deodorant N0 in purified water was 1.9.

### Example 57

The procedure of Example 45 was carried out in the same way except that the ZSM5 zeolite was used instead of the aluminum silicate, thus giving deodorant O0. The pH of a 5 wt % suspension of deodorant O0 in purified water was 1.9.

### Example 58

The procedure of Example 46 was carried out in the same way except that the ZSM5 zeolite was used instead of the aluminum silicate, thus giving deodorant P0. The pH of a 5 wt % suspension of deodorant P0 in purified water was 1.9.

### Example 59

The procedure of Example 43 was carried out in the same way except that the pH-adjusted mica was used instead of the aluminum silicate, thus giving deodorant Q0. The pH of a 5 wt % suspension of deodorant Q0 in purified water was 4.7.

### Example 60

The procedure of Example 44 was carried out in the same way except that the pH-adjusted mica was used instead of the aluminum silicate, thus giving deodorant R0. The pH of a 5 wt % suspension of deodorant R0 in purified water was 4.7.

### Example 61

The procedure of Example 45 was carried out in the same way except that the pH-adjusted mica was used instead of the aluminum silicate, thus giving deodorant S0. The pH of a 5 wt % suspension of deodorant S0 in purified water was 4.7.

### Example 62

The procedure of Example 46 was carried out in the same way except that the pH-adjusted mica was used instead of the aluminum silicate, thus giving deodorant T0. The pH of a 5 wt % suspension of deodorant T0 in purified water was 4.7.

### Example 63

The procedure of Example 23 was carried out in the same way except that, after the aluminum silicate was stirred at 100° C. for 1 hour, aminoguanidine hydrochloride was added thereto at 100° C. and mixed, thus giving deodorant A1 (100). The pH of a 5 wt % suspension of deodorant A1 (100) in purified water was 4.3.

### Example 64

The procedure of Example 24 was carried out in the same way except that, after the aluminum silicate was stirred at 100° C. for 1 hour, aminoguanidine sulfate was added thereto at 100° C. and mixed, thus giving deodorant B1 (100). The pH of a 5 wt % suspension of deodorant B1 (100) in purified water was 4.3.

### Example 65

70 parts by weight of deodorant A, 10 parts by weight of a layered α-zirconium phosphate, 10 parts by weight of a copper-binding layered α-zirconium phosphate, and 10 parts by weight of hydrated zirconium oxide were mixed well at room temperature, thus giving deodorant composition A.

### Example 66

The procedure of Example 65 was carried out in the same way except that deodorant B was used instead of deodorant A, thus giving deodorant composition B.

### Example 67

The procedure of Example 65 was carried out in the same way except that deodorant C was used instead of deodorant A, thus giving deodorant composition C.

### Example 68

The procedure of Example 65 was carried out in the same way except that deodorant D was used instead of deodorant A, thus giving deodorant composition D.

### Example 69

80 parts by weight of deodorant A and 20 parts by weight of zinc oxide were mixed well at room temperature, thus giving deodorant composition A′.

### Example 70

The procedure of Example 69 was carried out in the same way except that deodorant B was used instead of deodorant A, thus giving deodorant composition B′.

### Example 71

The procedure of Example 69 was carried out in the same way except that deodorant A (100) was used instead of deodorant A, thus giving deodorant composition A (100).

### Example 72

The procedure of Example 69 was carried out in the same way except that deodorant B (100) was used instead of deodorant A, thus giving deodorant composition B (100).

### Example 73

The procedure of Example 69 was carried out in the same way except that deodorant A1 (100) was used instead of deodorant A, thus giving deodorant composition A1 (100).

### Example 74

The procedure of Example 69 was carried out in the same way except that deodorant B1 (100) was used instead of deodorant A, thus giving deodorant composition B1 (100).

### Comparative Example 1

The procedure of Example 1 was carried out in the same way except that KW-2100 hydrotalcite (manufactured by Kyowa Chemical Industry Co., Ltd., the pH of a 5% suspension thereof in water was 10.5, the same material being used below) was used instead of the aluminum silicate, thus giving sample a. The pH of a 5 wt % suspension of sample a in purified water was 10.0.

### Comparative Example 2

The procedure of Example 2 was carried out in the same way except that the KW-2100 hydrotalcite was used instead of the aluminum silicate, thus giving sample b. The pH of a 5 wt % suspension of sample b in purified water was 10.0.

### Comparative Example 3

The procedure of Example 3 was carried out in the same way except that the KW-2100 hydrotalcite was used instead of the aluminum silicate, thus giving sample c. The pH of a 5 wt % suspension of sample c in purified water was 10.0.

### Comparative Example 4

The procedure of Example 4 was carried out in the same way except that the KW-2100 hydrotalcite was used instead of the aluminum silicate, thus giving sample d. The pH of a 5 wt % suspension of sample d in purified water was 10.0.

### Comparative Example 5

The procedure of Example 1 was carried out in the same way except that SOMASIF ME-100 synthetic swelling mica (manufactured by Co-op Chemical Co., Ltd., the pH of a 5% suspension thereof in water was 10.7) was used instead of the aluminum silicate, thus giving sample e. The pH of a 5 wt % suspension of sample e in purified water was 10.4.

### Comparative Example 6

The procedure of Example 2 was carried out in the same way except that the SOMASIF ME-100 synthetic swelling mica was used instead of the aluminum silicate, thus giving sample f. The pH of a 5 wt % suspension of sample f in purified water was 10.4.

### Comparative Example 7

The procedure of Example 3 was carried out in the same way except that the SOMASIF ME-100 synthetic swelling mica was used instead of the aluminum silicate, thus giving sample g. The pH of a 5 wt % suspension of sample g in purified water was 10.4.

### Comparative Example 8

The procedure of Example 4 was carried out in the same way except that the SOMASIF ME-100 synthetic swelling mica was used instead of the aluminum silicate, thus giving sample h. The pH of a 5 wt % suspension of sample h in purified water was 10.4.

Preparation of pH-Adjusted Aluminum Silicate B

While stirring 100 parts by weight of the aluminum silicate (the pH of a 5 wt % suspension thereof in water was 6.5), 50 parts by weight of a 10% sodium hydroxide aqueous solution was added thereto at room temperature, and mixed well, thus giving pH-adjusted aluminum silicate B.

### Comparative Example 9

The procedure of Example 1 was carried out in the same way except that the pH-adjusted aluminum silicate B was used instead of the aluminum silicate, thus giving sample i. The pH of a 5 wt % suspension of sample i in purified water was 10.7.

### Comparative Example 10

The procedure of Example 2 was carried out in the same way except that the pH-adjusted aluminum silicate B was used instead of the aluminum silicate, thus giving sample j. The pH of a 5 wt % suspension of sample j in purified water was 10.7.

### Comparative Example 11

The procedure of Example 3 was carried out in the same way except that the pH-adjusted aluminum silicate B was used instead of the aluminum silicate, thus giving sample k. The pH of a 5 wt % suspension of sample k in purified water was 10.7.

### Comparative Example 12

The procedure of Example 4 was carried out in the same way except that the pH-adjusted aluminum silicate B was used instead of the aluminum silicate, thus giving sample l. The pH of a 5 wt % suspension of sample l in purified water was 10.7.

### Comparative Example 13

The procedure of Example 23 was carried out in the same way except that the KW-2100 hydrotalcite was used instead of the aluminum silicate, thus giving sample m. The pH of a 5 wt % suspension of sample m in purified water was 9.5.

### Comparative Example 14

The procedure of Example 24 was carried out in the same way except that the KW-2100 hydrotalcite was used instead of the aluminum silicate, thus giving sample n. The pH of a 5 wt % suspension of sample n in purified water was 9.5.

### Comparative Example 15

The procedure of Example 25 was carried out in the same way except that the KW-2100 hydrotalcite was used instead of the aluminum silicate, thus giving sample o. The pH of a 5 wt % suspension of sample o in purified water was 9.5.

### Comparative Example 16

The procedure of Example 26 was carried out in the same way except that the KW-2100 hydrotalcite was used instead of the aluminum silicate, thus giving sample p. The pH of a 5 wt % suspension of sample p in purified water was 9.5.

### Comparative Example 17

The procedure of Example 23 was carried out in the same way except that the SOMASIF ME-100 synthetic swelling mica was used instead of the aluminum silicate, thus giving sample q. The pH of a 5 wt % suspension of sample q in purified water was 9.7.

### Comparative Example 18

The procedure of Example 24 was carried out in the same way except that the SOMASIF ME-100 synthetic swelling mica was used instead of the aluminum silicate, thus giving sample r. The pH of a 5 wt % suspension of sample r in purified water was 9.7.

### Comparative Example 19

The procedure of Example 25 was carried out in the same way except that the SOMASIF ME-100 synthetic swelling mica was used instead of the aluminum silicate, thus giving sample s. The pH of a 5 wt % suspension of sample s in purified water was 9.7.

### Comparative Example 20

The procedure of Example 26 was carried out in the same way except that the SOMASIF ME-100 synthetic swelling mica was used instead of the aluminum silicate, thus giving sample t. The pH of a 5 wt % suspension of sample t in purified water was 9.7.

### Comparative Example 21

The procedure of Example 23 was carried out in the same way except that the pH-adjusted aluminum silicate B was used instead of the aluminum silicate, thus giving sample u. The pH of a 5 wt % suspension of sample u in purified water was 10.2.

### Comparative Example 22

The procedure of Example 24 was carried out in the same way except that the pH-adjusted aluminum silicate B was used instead of the aluminum silicate, thus giving sample v. The pH of a 5 wt % suspension of sample v in purified water was 10.2.

### Comparative Example 23

The procedure of Example 25 was carried out in the same way except that the pH-adjusted aluminum silicate B was used instead of the aluminum silicate, thus giving sample w. The pH of a 5 wt % suspension of sample w in purified water was 10.2.

### Comparative Example 24

The procedure of Example 26 was carried out in the same way except that the pH-adjusted aluminum silicate B was used instead of the aluminum silicate, thus giving sample x. The pH of a 5 wt % suspension of sample x in purified water was 10.2.

### Comparative Example 25

70 parts by weight of sample a, 10 parts by weight of layered α-zirconium phosphate, 10 parts by weight of copper-binding layered α-zirconium phosphate, and 10 parts by weight of hydrated zirconium oxide were mixed well at room temperature, thus giving sample composition a.

### Comparative Example 26

The procedure of Comparative Example 25 was carried out in the same way except that sample b was used instead of sample a, thus giving sample composition b.

### Comparative Example 27

The procedure of Comparative Example 25 was carried out in the same way except that sample c was used instead of sample a, thus giving sample composition c.

### Comparative Example 28

The procedure of Comparative Example 25 was carried out in the same way except that sample d was used instead of sample a, thus giving sample composition d.

### Example 75

### Test for Water Resistance of Deodorant

After the deodorants prepared in the Examples were washed with purified water, the deodorizing activity toward acetaldehyde gas was measured. That is, 1 g of deodorant A was placed in 100 mL of purified water at room temperature and stirred well for 1 minute. After this liquid was filtered, washing was further carried out with 1000 mL of purified water, and drying was carried out at 110° C. Deodorant A thus washed with water (after washing with water) was subjected to measurement of the deodorizing activity toward acetaldehyde gas. Similarly, with regard to the other deodorants, deodorizing activity was measured for those that had been washed with water in this way. The samples prepared in the Comparative Examples were also washed with water in the same manner and subjected to measurement of the deodorizing activity.

### Example 76

### Measurement of Deodorizing Effect of Wet-Supported Deodorant

Measurement of the deodorizing effect of deodorant A prepared in Example 1 involved placing 0.02 g thereof in a vinyl fluoride bag (a vinyl fluoride film was used in the form of a bag, this hereinafter being called a Tedlar bag), injecting thereinto 1 L of air containing 800 ppm of acetaldehyde gas, and allowing it to stand at room temperature or 80° C. for 2 hours. After 2 hours, the concentration of acetaldehyde gas remaining in the Tedlar bag was measured by a gas detector tube (manufactured by Gastec Corporation, the same company's product being used below). The measurement results are given in Table 1. Furthermore, deodorant A that had been subjected to the water resistance test (after washing with water) was also tested, and the results are given in Table 1.

Moreover, the acetaldehyde gas deodorizing effect was measured in the same manner for the deodorants prepared in the Examples and the samples prepared in the Comparative Examples, and the results are given in Table 2.

Furthermore, when 1 mg of aminoguanidine hydrochloride, aminoguanidine sulfate, diaminoguannidine hydrochloride, or triaminoguanidine hydrochloride was placed directly in this evaluation system and the deodorizing activity was measured, the concentration of acetaldehyde was 22 ppm, 24 ppm, 10 ppm, or 8 ppm respectively when left to stand at room temperature, and 275 ppm, 275 ppm, 225 ppm, or 175 ppm respectively when left to stand at 80° C.

TABLE 1

| | Left to stand at | | | |
|---|---|---|---|---|
| | room temperature | | Left to stand at 80° C. | |
| | Before | After | Before | After |
| | washing | washing | washing | washing |
| | with water | with water | with water | with water |
| Deodorant A | <0.2 | 10 | 4 | 50 |
| Deodorant B | <0.2 | 10 | 4 | 50 |
| Deodorant C | <0.2 | 2 | 2 | 40 |
| Deodorant D | <0.2 | 3 | 2 | 40 |
| Deodorant E | <0.2 | 14 | 6 | 55 |
| Deodorant F | <0.2 | 12 | 6 | 55 |
| Deodorant G | <0.2 | 4 | 4 | 50 |
| Deodorant H | <0.2 | 4 | 4 | 50 |
| Deodorant I | <0.2 | 12 | 4 | 50 |
| Deodorant J | <0.2 | 12 | 4 | 50 |
| Deodorant K | <0.2 | 6 | 2 | 50 |
| Deodorant L | <0.2 | 6 | 2 | 50 |
| Deodorant M | <0.2 | 15 | 4 | 55 |
| Deodorant N | <0.2 | 15 | 4 | 55 |
| Deodorant O | <0.2 | 6 | 2 | 50 |
| Deodorant P | <0.2 | 6 | 2 | 50 |
| Deodorant Q | 10 | 30 | 175 | 275 |
| Deodorant R | 10 | 30 | 175 | 275 |
| Deodorant S | 2 | 25 | 150 | 250 |
| Deodorant T | 2 | 25 | 150 | 250 |
| Deodorant A (100) | <0.2 | 10 | 4 | 50 |
| Deodorant B (100) | <0.2 | 10 | 4 | 50 |

TABLE 2

| | |
|---|---|
| Left to stand at | |
| room temperature | Left to stand at 80° C. |

| | Before | After | Before | After |
|---|---|---|---|---|
| | washing | washing | washing | washing |
| | with water | with water | with water | with water |
| Sample a | 375 | 525 | 575 | 725 |
| Sample b | 375 | 525 | 575 | 725 |
| Sample c | 325 | 425 | 475 | 675 |
| Sample d | 325 | 425 | 475 | 675 |
| Sample e | 375 | 525 | 575 | 725 |
| Sample f | 375 | 525 | 575 | 725 |
| Sample g | 325 | 425 | 475 | 675 |
| Sample h | 325 | 425 | 475 | 675 |
| Sample i | 325 | 425 | 425 | 725 |
| Sample j | 325 | 425 | 425 | 725 |
| Sample k | 275 | 375 | 425 | 675 |
| Sample l | 275 | 375 | 425 | 675 |

From the results above, the aldehyde gas deodorant of the present invention has excellent acetaldehyde deodorizing performance, and also has excellent deodorizing performance after the water resistance test is carried out. Furthermore, in the present invention, those employing an inorganic powder having a pH of 2 to 8 when dispersed in water have excellent acetaldehyde deodorizing performance at 80° C. in particular, and have excellent deodorizing performance after the water resistance test is carried out. On the other hand, it has been found that the Comparative Examples have inferior acetaldehyde deodorizing performance to that of the Examples.

From this, the aldehyde gas deodorant of the present invention exhibits excellent deodorizing activity, and there is hardly any deterioration in the deodorizing activity even after carrying out washing with water. Furthermore, the aldehyde gas deodorant of the present invention has excellent deodorizing efficiency in a range from room temperature to high temperature. This suggests that the aldehyde gas deodorant of the present invention has excellent durability and deodorizing properties.

### Example 77

### Measurement of Deodorizing Effect of Dry-Supported Deodorant

Measurement of the deodorizing effect of deodorant A1 involved placing 0.1 g thereof in a Tedlar bag, and injecting thereinto 1 L of air containing 600 ppm of acetaldehyde. After allowing it to stand at room temperature or 80° C. for 2 hours, the concentration of acetaldehyde remaining in the Tedlar bag was measured by a gas detector. The results are given in Table 3. Furthermore, deodorant A1 after the water resistance test (after washing with water) was tested in the same manner.

Moreover, the deodorants prepared in the Examples and the samples prepared in the Comparative Examples were subjected to the same measurement of the acetaldehyde gas deodorizing effect, and the results are given in Tables 3 to 5.

Furthermore, when 5 mg of aminoguanidine hydrochloride, aminoguanidine sulfate, diaminoguannidine hydrochloride, or triaminoguanidine hydrochloride was placed directly in this evaluation system and the deodorizing activity was measured, the concentration of acetaldehyde was 3 ppm, 4 ppm, 1 ppm, or 1 ppm respectively when left to stand at room temperature, and 65 ppm, 65 ppm, 55 ppm, or 55 ppm respectively when left to stand at 80° C.

TABLE 3

| | Left to stand at | | | |
|---|---|---|---|---|
| | room temperature | | Left to stand at 80° C. | |
| | Before | After | Before | After |
| | washing | washing | washing | washing |
| | with water | with water | with water | with water |
| Deodorant A1 | <0.1 | 55 | 2 | 70 |
| Deodorant B1 | <0.1 | 60 | 2 | 70 |
| Deodorant C1 | <0.1 | 35 | 1 | 60 |
| Deodorant D1 | <0.1 | 35 | 1 | 60 |
| Deodorant E1 | <0.1 | 60 | 2 | 75 |
| Deodorant F1 | <0.1 | 60 | 2 | 75 |
| Deodorant G1 | <0.1 | 40 | 2 | 70 |
| Deodorant H1 | <0.1 | 38 | 2 | 70 |
| Deodorant I1 | <0.1 | 60 | 2 | 70 |
| Deodorant J1 | <0.1 | 60 | 2 | 70 |
| Deodorant K1 | <0.1 | 35 | 1 | 70 |
| Deodorant L1 | <0.1 | 35 | 1 | 70 |
| Deodorant M1 | <0.1 | 110 | 2 | 75 |
| Deodorant N1 | <0.1 | 120 | 2 | 75 |
| Deodorant O1 | <0.1 | 82 | 2 | 70 |
| Deodorant P1 | <0.1 | 80 | 2 | 70 |
| Deodorant Q1 | <0.1 | 175 | 35 | 325 |
| Deodorant R1 | <0.1 | 175 | 35 | 325 |
| Deodorant S1 | <0.1 | 135 | 30 | 275 |
| Deodorant T1 | <0.1 | 135 | 30 | 275 |
| Deodorant A1 (100) | <0.1 | 55 | 2 | 70 |
| Deodorant B1 (100) | <0.1 | 60 | 2 | 70 |

TABLE 4

| | Left to stand at | | | |
|---|---|---|---|---|
| | room temperature | | Left to stand at 80° C. | |
| | Before | After | Before | After |
| | washing | washing | washing | washing |
| | with water | with water | with water | with water |
| Deodorant A0 | <0.1 | 325 | 25 | 425 |
| Deodorant B0 | <0.1 | 325 | 25 | 425 |
| Deodorant C0 | <0.1 | 220 | 15 | 325 |
| Deodorant D0 | <0.1 | 220 | 15 | 325 |
| Deodorant E0 | <0.1 | 325 | 25 | 425 |
| Deodorant F0 | <0.1 | 350 | 25 | 425 |
| Deodorant G0 | <0.1 | 280 | 20 | 375 |
| Deodorant H0 | <0.1 | 260 | 20 | 375 |
| Deodorant I0 | <0.1 | 325 | 25 | 425 |
| Deodorant J0 | <0.1 | 325 | 25 | 425 |
| Deodorant K0 | <0.1 | 245 | 15 | 375 |
| Deodorant L0 | <0.1 | 245 | 15 | 375 |
| Deodorant M0 | <0.1 | 425 | 25 | 475 |
| Deodorant N0 | <0.1 | 475 | 25 | 475 |
| Deodorant O0 | <0.1 | 370 | 15 | 425 |
| Deodorant P0 | <0.1 | 350 | 15 | 425 |
| Deodorant Q0 | 1 | 425 | 40 | 450 |
| Deodorant R0 | 1 | 425 | 40 | 450 |
| Deodorant S0 | 2 | 375 | 35 | 425 |
| Deodorant T0 | 2 | 375 | 35 | 425 |

TABLE 5

| | |
|---|---|
| Left to stand at | |
| room temperature | Left to stand at 80° C. |

| | Before | After | Before | After |
|---|---|---|---|---|
| | washing | washing | washing | washing |
| | with water | with water | with water | with water |
| Sample m | 375 | 525 | 475 | 525 |
| Sample n | 375 | 525 | 475 | 525 |
| Sample o | 325 | 425 | 475 | 525 |
| Sample p | 325 | 425 | 475 | 525 |
| Sample q | 375 | 525 | 475 | 525 |
| Sample r | 375 | 525 | 475 | 525 |
| Sample s | 325 | 425 | 475 | 525 |
| Sample t | 325 | 425 | 475 | 525 |
| Sample u | 325 | 425 | 425 | 525 |
| Sample v | 325 | 425 | 425 | 525 |
| Sample w | 275 | 375 | 425 | 525 |
| Sample x | 275 | 375 | 425 | 525 |

From the results above, when the aminoguanidine salt and the inorganic powder are mixed and then heated, compared with one that is not heated, deterioration of the deodorizing activity by washing with water is suppressed, and the deodorizing efficiency is excellent. Furthermore, in the present invention, when an inorganic powder having a pH of 2 to 8 when dispersed in water is used, the acetaldehyde deodorizing performance at 80° C. in particular is excellent, and the deodorizing performance after carrying out the water resistance test is also excellent. On the other hand, it has been found that the Comparative Examples have inferior acetaldehyde deodorizing performance to that of the Examples.

From this, the aldehyde gas deodorant of the present invention exhibits excellent deodorizing activity, and when it is heated there is hardly any deterioration in the deodorizing activity even after carrying out washing with water. Furthermore, the aldehyde gas deodorant of the present invention has excellent deodorizing efficiency. This suggests that the aldehyde gas deodorant of the present invention has excellent durability and deodorizing properties.

### Example 78

### Test for Water Resistance of Deodorant Composition

After the deodorant composition was washed with purified water, the deodorizing activity toward acetaldehyde gas was measured. That is, 1 g of deodorant composition A was placed in 100 mL of purified water at room temperature and stirred well for 1 minute. After this liquid was filtered, washing was further carried out with 1000 mL of purified water, and drying was carried out at 110° C. Deodorant composition A thus washed with water was subjected to measurement of the deodorizing activity toward acetaldehyde gas, ammonia gas, hydrogen sulfide gas, and acetic acid gas. Similarly, with regard to the other deodorant compositions, the deodorizing activity was measured for those that had been washed with water in this way. The sample compositions prepared in the Comparative Examples were also washed with water in the same manner and subjected to measurement of the deodorizing activity.

### Measurement of Deodorizing Effect of Deodorant Composition

Measurement of the deodorizing effect involved placing 0.02 g of each of the samples subjected to the above water resistance test in a corresponding Tedlar bag, injecting thereinto 1 L of air containing 20 ppm of acetaldehyde gas, 40 ppm of ammonia gas, 10 ppm of hydrogen sulfide gas, and 40 ppm of acetic acid gas, and allowing it to stand at room temperature. After 2 hours, the concentration of each gas remaining in the Tedlar bag was measured by a corresponding gas detector tube. The results are given in Table 6.

TABLE 6

| | Ammo- | Acetal- | Hydrogen | Acetic |
|---|---|---|---|---|
| | nia | dehyde | sulfide | acid |
| Deodorant composition A | <0.2 | <0.2 | 6 | 2 |
| Deodorant composition B | <0.2 | <0.2 | 6 | 2 |
| Deodorant composition C | <0.2 | <0.2 | 6 | 2 |
| Deodorant composition D | <0.2 | <0.2 | 6 | 2 |
| Deodorant composition A′ | <0.2 | <0.2 | <0.1 | 2 |
| Deodorant composition B′ | <0.2 | <0.2 | <0.1 | 2 |
| Deodorant composition A (100) | <0.2 | <0.2 | <0.1 | 2 |
| Deodorant composition B (100) | <0.2 | <0.2 | <0.1 | 2 |
| Deodorant composition A1 (100) | <0.2 | <0.2 | <0.1 | 2 |
| Deodorant composition B1 (100) | <0.2 | <0.2 | <0.1 | 2 |
| Sample composition a | <0.2 | 18 | 8 | 6 |
| Sample composition b | <0.2 | 18 | 8 | 6 |
| Sample composition c | <0.2 | 18 | 8 | 6 |
| Sample composition d | <0.2 | 18 | 9 | 6 |

It has been found that the deodorant composition of the present invention in which the aldehyde gas deodorant of the present invention and another bad odor deodorant are mixed has high deodorizing performance for acetaldehyde, hydrogen sulfide, acetic acid, etc. compared with the Comparative Examples.

### Comparative Example 29

The procedure of Example 1 was carried out in the same way except that 50 parts by weight of an 8% adipic acid dihydrazide aqueous solution was used instead of the aminoguanidine hydrochloride aqueous solution, thus giving sample 1a. The deodorizing effect of this sample was measured in accordance with the deodorizing effect measurement methods in Example 78 and Example 79, and the results are given in Table 7.

### Comparative Example 30

The procedure of Example 5 was carried out in the same way except that 50 parts by weight of an 8% adipic acid dihydrazide aqueous solution was used instead of the aminoguanidine hydrochloride aqueous solution, thus giving sample 1b. The deodorizing effect of this sample was measured in accordance with the deodorizing effect measurement methods in Example 78 and Example 79, and the results are given in Table 7.

### Comparative Example 31

The procedure of Example 9 was carried out in the same way except that 50 parts by weight of an 8% adipic acid dihydrazide aqueous solution was used instead of the aminoguanidine hydrochloride aqueous solution, thus giving sample 1c. The deodorizing effect of this sample was measured in accordance with the deodorizing effect measurement methods in Example 78 and Example 79, and the results are given in Table 7.

### Comparative Example 32

The procedure of Example 1 was carried out in the same way except that 50 parts by weight of a 30% guanidine hydrochloride aqueous solution was used instead of the aminoguanidine hydrochloride aqueous solution, thus giving sample 1d. The deodorizing effect of this sample was measured in accordance with the deodorizing effect measurement methods in Example 78 and Example 79, and the results are given in Table 7.

### Comparative Example 33

The procedure of Example 5 was carried out in the same way except that 50 parts by weight of a 30% guanidine hydrochloride aqueous solution was used instead of the aminoguanidine hydrochloride aqueous solution, thus giving sample 1e. The deodorizing effect of this sample was measured in accordance with the deodorizing effect measurement methods in Example 78 and Example 79, and the results are given in Table 7.

### Comparative Example 34

The procedure of Example 9 was carried out in the same way except that 50 parts by weight of a 30% guanidine hydrochloride aqueous solution was used instead of the aminoguanidine hydrochloride aqueous solution, thus giving sample 1f. The deodorizing effect of this sample was measured in accordance with the deodorizing effect measurement methods in Example 78 and Example 79, and the results are given in Table 7.

### Comparative Example 35

The procedure of Example 1 was carried out in the same way except that 50 parts by weight of a 30% urea aqueous solution was used instead of the aminoguanidine hydrochloride aqueous solution, thus giving sample 1 g. The deodorizing effect of this sample was measured in accordance with the deodorizing effect measurement methods in Example 78 and Example 79, and the results are given in Table 7.

### Comparative Example 36

The procedure of Example 5 was carried out in the same way except that 50 parts by weight of a 30% urea aqueous solution was used instead of the aminoguanidine hydrochloride aqueous solution, thus giving sample 1 h. The deodorizing effect of this sample was measured in accordance with the deodorizing effect measurement methods in Example 78 and Example 79, and the results are given in Table 7.

### Comparative Example 37

The procedure of Example 9 was carried out in the same way except that 50 parts by weight of a 30% urea aqueous solution was used instead of the aminoguanidine hydrochloride aqueous solution, thus giving sample 11. The deodorizing effect of this sample was measured in accordance with the deodorizing effect measurement methods in Example 78 and Example 79, and the results are given in Table 7.

TABLE 7

| | Before washing with water | After washing with water |
|---|---|---|
| Sample 1a | 375 | 525 |
| Sample 1b | 400 | 525 |
| Sample 1c | 325 | 525 |
| Sample 1d | 725 | 750 |
| Sample 1e | 725 | 750 |
| Sample 1f | 725 | 750 |
| Sample 1g | 475 | 625 |
| Sample 1h | 475 | 625 |
| Sample 1i | 475 | 625 |

### Example 80

### Preparation of Deodorant Dispersion

50 parts of deodorant A, 2 parts of Disperbyk-180 dispersant (alkyl ammonium salt of a phosphoric acid group-containing block copolymer, acid value 94 mg KOH/g, amine value 94 mg KOH/g, average molecular weight 1000, manufactured by BYK-Chemie), 0.3 parts of Bestcide #300 preservative (manufactured by Dainippon Ink and Chemicals, Incorporated), 0.2 parts of Disperbyk-022 antifoaming agent (manufactured by BYK-Chemie Japan), and 5 parts of a 4% aqueous solution of Metholose SH15000 thickener (manufactured by Shin-Etsu Chemical Co., Ltd.) were added to 100 parts of purified water and stirred using a sand mill at 3000 rpm for 20 minutes, thus giving deodorant dispersion A.

### Example 81

The procedure of Example 80 was carried out in the same way except that deodorant B was used instead of deodorant A, thus giving deodorant dispersion B.

### Example 82

The procedure of Example 80 was carried out in the same way except that deodorant C was used instead of deodorant A, thus giving deodorant dispersion C.

### Example 83

The procedure of Example 80 was carried out in the same way except that deodorant D was used instead of deodorant A, thus giving deodorant dispersion D.

### Example 84

The procedure of Example 80 was carried out in the same way except that deodorant E was used instead of deodorant A, thus giving deodorant dispersion E.

### Example 85

The procedure of Example 80 was carried out in the same way except that deodorant F was used instead of deodorant A, thus giving deodorant dispersion F.

### Example 86

The procedure of Example 80 was carried out in the same way except that deodorant G was used instead of deodorant A, thus giving deodorant dispersion G.

### Example 87

The procedure of Example 80 was carried out in the same way except that deodorant H was used instead of deodorant A, thus giving deodorant dispersion H.

### Example 88

The procedure of Example 80 was carried out in the same way except that deodorant I was used instead of deodorant A, thus giving deodorant dispersion I.

### Example 89

The procedure of Example 80 was carried out in the same way except that deodorant J was used instead of deodorant A, thus giving deodorant dispersion J.

### Example 90

The procedure of Example 80 was carried out in the same way except that deodorant K was used instead of deodorant A, thus giving deodorant dispersion K.

### Example 91

The procedure of Example 80 was carried out in the same way except that deodorant L was used instead of deodorant A, thus giving deodorant dispersion L.

### Example 92

The procedure of Example 80 was carried out in the same way except that deodorant M was used instead of deodorant A, thus giving deodorant dispersion M.

### Example 93

The procedure of Example 80 was carried out in the same way except that deodorant N was used instead of deodorant A, thus giving deodorant dispersion N.

### Example 94

The procedure of Example 80 was carried out in the same way except that deodorant O was used instead of deodorant A, thus giving deodorant dispersion O.

### Example 95

The procedure of Example 80 was carried out in the same way except that deodorant P was used instead of deodorant A, thus giving deodorant dispersion P.

### Example 96

50 parts of deodorant B, 2 parts of Disperbyk-180 dispersant (alkylammonium salt of a phosphoric acid group-containing block copolymer, acid value 94 mg KOH/g, amine value 94 mg KOH/g, average molecular weight 1000, manufactured by BYK-Chemie), 0.3 parts of Bestcide #300 preservative (manufactured by Dainippon Ink and Chemicals, Incorporated), 0.2 parts of Disperbyk-022 antifoaming agent (manufactured by BYK-Chemie Japan), 5 parts of a 4% aqueous solution of Metholose SH15000 thickener (manufactured by Shin-Etsu Chemical Co., Ltd.), and 1 part of polyethylene glycol 400 (manufactured by Wako Pure Chemical Industries, Ltd., average molecular weight 400) were added to 100 parts of purified water and stirred using a sand mill at 3000 rpm for 20 minutes, thus giving deodorant dispersion B (p1).

### Example 97

The procedure of Example 96 was carried out in the same way except that deodorant F was used instead of deodorant B, thus giving deodorant dispersion F (p1).

### Example 98

The procedure of Example 96 was carried out in the same way except that deodorant J was used instead of deodorant B, thus giving deodorant dispersion J (p1).

### Example 99

The procedure of Example 96 was carried out in the same way except that polyethylene glycol 6000 (manufactured by Wako Pure Chemical Industries, Ltd., average molecular weight 7300 to 9300) was used instead of polyethylene glycol 400, thus giving deodorant dispersion B (p2).

### Example 100

The procedure of Example 97 was carried out in the same way except that polyethylene glycol 6000 (manufactured by Wako Pure Chemical Industries, Ltd., average molecular weight 7300 to 9300) was used instead of polyethylene glycol 400, thus giving deodorant dispersion F (p2).

### Example 101

The procedure of Example 98 was carried out in the same way except that polyethylene glycol 6000 (manufactured by Wako Pure Chemical Industries, Ltd., average molecular weight 7300 to 9300) was used instead of polyethylene glycol 400, thus giving deodorant dispersion J (p2).

### Example 102

The procedure of Example 80 was carried out in the same way except that deodorant composition A was used instead of deodorant A, thus giving deodorant composition dispersion A.

### Example 103

The procedure of Example 80 was carried out in the same way except that deodorant composition B was used instead of deodorant A, thus giving deodorant composition dispersion B.

### Example 104

The procedure of Example 80 was carried out in the same way except that deodorant composition C was used instead of deodorant A, thus giving deodorant composition dispersion C.

### Example 105

The procedure of Example 80 was carried out in the same way except that deodorant composition D was used instead of deodorant A, thus giving deodorant composition dispersion D.

### Example 106

The procedure of Example 80 was carried out in the same way except that deodorant composition A′ was used instead of deodorant A, thus giving deodorant composition dispersion A′.

### Example 107

The procedure of Example 80 was carried out in the same way except that deodorant composition B′ was used instead of deodorant A, thus giving deodorant composition dispersion B′.

### Example 108

The procedure of Example 80 was carried out in the same way except that deodorant composition A (100) was used instead of deodorant A, thus giving deodorant composition dispersion A (100).

### Example 109

The procedure of Example 80 was carried out in the same way except that deodorant composition B (100) was used instead of deodorant A, thus giving deodorant composition dispersion B (100).

### Example 110

The procedure of Example 80 was carried out in the same way except that deodorant composition A1 (100) was used instead of deodorant A, thus giving deodorant composition dispersion A1 (100).

### Example 111

The procedure of Example 80 was carried out in the same way except that deodorant composition B1 (100) was used instead of deodorant A, thus giving deodorant composition dispersion B1 (100).

### Example 112

The procedure of Example 80 was carried out in the same way except that polyoxyethylene nonylphenyl ether (nonionic dispersant) was used instead of Disperbyk-180 dispersant, thus giving deodorant dispersion A1.

### Example 113

The procedure of Example 80 was carried out in the same way except that sodium hexametaphosphate (inorganic dispersant) was used instead of Disperbyk-180 dispersant, thus giving deodorant dispersion A2.

### Example 114

The procedure of Example 80 was carried out in the same way except that the dispersant was added at 10 parts (20 parts relative to 100 parts of the deodorant) relative to 100 parts of water, thus giving deodorant dispersion A3.

### Example 115

The procedure of Example 80 was carried out in the same way except that the dispersant was added at 0.015 parts (0.03 parts relative to 100 parts of the deodorant) relative to 100 parts of water, thus giving deodorant dispersion A4.

### Example 116

### Evaluation of Stability of Each Dispersion

The dispersibility of each dispersion prepared in the Examples and Comparative Examples was visually examined after allowing it to stand at room temperature for 24 hours. The results are given in Table 8.

Furthermore, each dispersion was placed in a 1 L polyethylene bottle and left to stand at 30° C. for 1 month. Following this, the height of the supernatant was measured, and the sedimentation properties were evaluated. The results are given in Table 8.

TABLE 8

| | | Height of | |
|---|---|---|---|
| | Visual | supernatant | |
| | examination | portion | |
| Deodorant dispersion A | Good | 5 | mm |
| Deodorant dispersion B | Good | 5 | mm |
| Deodorant dispersion C | Good | 5 | mm |
| Deodorant dispersion D | Good | 5 | mm |
| Deodorant dispersion E | Good | 5 | mm |
| Deodorant dispersion F | Good | 5 | mm |
| Deodorant dispersion G | Good | 5 | mm |
| Deodorant dispersion H | Good | 5 | mm |
| Deodorant dispersion I | Good | 5 | mm |
| Deodorant dispersion J | Good | 5 | mm |
| Deodorant dispersion K | Good | 5 | mm |
| Deodorant dispersion L | Good | 5 | mm |
| Deodorant dispersion M | Good | 8 | mm |
| Deodorant dispersion N | Good | 8 | mm |
| Deodorant dispersion O | Good | 8 | mm |
| Deodorant dispersion P | Good | 8 | mm |
| Deodorant dispersion B (p1) | Good | 5 | mm |
| Deodorant dispersion F (p1) | Good | 5 | mm |
| Deodorant dispersion J (p1) | Good | 5 | mm |
| Deodorant dispersion B (p2) | Good | 5 | mm |
| Deodorant dispersion F (p2) | Good | 5 | mm |
| Deodorant dispersion J (p2) | Good | 5 | mm |
| Deodorant composition | Good | 5 | mm |
| dispersion A | | | |
| Deodorant composition | Good | 5 | mm |
| dispersion B | | | |
| Deodorant composition | Good | 5 | mm |
| dispersion C | | | |
| Deodorant composition | Good | 5 | mm |
| dispersion D | | | |
| Deodorant composition | Good | 5 | mm |
| dispersion A′ | | | |
| Deodorant composition | Good | 5 | mm |
| dispersion B′ | | | |
| Deodorant composition | Good | 5 | mm |
| dispersion A (100) | | | |
| Deodorant composition | Good | 5 | mm |
| dispersion B (100) | | | |
| Deodorant composition | Good | 5 | mm |
| dispersion A1 (100) | | | |
| Deodorant composition | Good | 5 | mm |
| dispersion B1 (100) | | | |
| Deodorant dispersion A1 | Small amount of precipitate | 20 | mm |
| Deodorant dispersion A2 | Small amount of precipitate | 20 | mm |
| Deodorant dispersion A3 | No flowability due to | 0 | mm |
| | increase in viscosity | | |
| Deodorant dispersion A4 | Precipitate | 20 | mm |

This suggests that the dispersibility is good when the amount of dispersant in the deodorant dispersion of the present invention is 2 to 10 parts relative to 100 parts of the deodorant solids content. However, even when the amount of dispersant was 12 parts, the dispersion did not become poor. Nevertheless, the dispersion becomes poor if the amount of dispersant is too large or too small.

### Example 117

### Test for Discoloration of Each Dispersion

The discoloration of dispersions described in Table 9, such as deodorant composition dispersion A′ and deodorant composition dispersion B′, was examined by measuring the color immediately after preparing the dispersions and after storing the dispersions at 50° C. for 24 hours. The results are given in Table 9. The method for measuring color involved placing 5 mL of each dispersion in a 9 mL capacity Laboran screw-top vial (material: borosilicate glass, manufactured by AS ONE Corporation), and measuring the color (L, a, b) using a colorimeter (SZ-Σ80 colorimeter, manufactured by Nippon Denshoku Industries Co., Ltd.). In this process, light from the colorimeter was applied from the bottom of the Laboran screw-top vial. A color difference ΔE was determined by comparing the color immediately after preparing the dispersions with that after storing at 50° C. for 24 hours. The results are given in Table 9.

TABLE 9

| | Immediately after | | | After storage at | | | |
|---|---|---|---|---|---|---|---|
| | dispersion preparation | | | 50° C. for 24 hours | | | |
| | L | a | b | L | a | b | ΔE |
| Deodorant composition | 82.5 | −3.3 | 3.1 | 82.8 | −2.4 | 2.9 | 1.0 |
| dispersion A′ | | | | | | | |
| Deodorant composition | 81.3 | −3.2 | 2.9 | 81.8 | −2.2 | 2.8 | 1.1 |
| dispersion B′ | | | | | | | |
| Deodorant composition | 80.3 | −2.9 | 3.0 | 80.0 | 1.5 | 3.1 | 4.4 |
| dispersion A (100) | | | | | | | |
| Deodorant composition | 78.9 | −2.8 | 2.8 | 78.6 | 1.1 | 2.6 | 3.9 |
| dispersion B (100) | | | | | | | |
| Deodorant composition | 80.8 | −3.2 | 3.0 | 80.2 | 1.0 | 3.0 | 4.2 |
| dispersion A1 (100) | | | | | | | |
| Deodorant composition | 79.4 | −3.0 | 2.9 | 78.9 | 0.9 | 2.9 | 3.9 |
| dispersion B1 (100) | | | | | | | |

The deodorant of the present invention in which the aminoguanidine salt was added at a temperature greater than 60° C. showed a high degree of discoloration when stored as a deodorant composition dispersion, compared with one in which the aminoguanidine salt was added at less than 60° C.

### Example 118

### Preparation of Deodorizing Fiber

A suspension was prepared by adding 10 parts by weight of deodorant dispersion A and 3 parts by weight of an acrylic binder (KB-1300, manufactured by Toagosei Co., Ltd.) to 100 parts by weight of purified water. 100 parts by weight of polyester fiber was coated with 50 parts by weight of the suspension and dried at 150° C., thus giving deodorizing fiber A (the content of the deodorant was 1.5 parts relative to 100 parts by weight of resin).

### Example 119

The procedure of Example 118 was carried out in the same way except that deodorant dispersion B was used instead of deodorant dispersion A, thus giving deodorizing fiber B.

### Example 120

The procedure of Example 118 was carried out in the same way except that deodorant dispersion C was used instead of deodorant dispersion A, thus giving deodorizing fiber C.

### Example 121

The procedure of Example 118 was carried out in the same way except that deodorant dispersion D was used instead of deodorant dispersion A, thus giving deodorizing fiber D.

### Example 122

The procedure of Example 118 was carried out in the same way except that deodorant dispersion E was used instead of deodorant dispersion A, thus giving deodorizing fiber E.

### Example 123

The procedure of Example 118 was carried out in the same way except that deodorant dispersion F was used instead of deodorant dispersion A, thus giving deodorizing fiber F.

### Example 124

The procedure of Example 118 was carried out in the same way except that deodorant dispersion G was used instead of deodorant dispersion A, thus giving deodorizing fiber G.

### Example 125

The procedure of Example 118 was carried out in the same way except that deodorant dispersion H was used instead of deodorant dispersion A, thus giving deodorizing fiber H.

### Example 126

The procedure of Example 118 was carried out in the same way except that deodorant dispersion I was used instead of deodorant dispersion A, thus giving deodorizing fiber I.

### Example 127

The procedure of Example 118 was carried out in the same way except that deodorant dispersion J was used instead of deodorant dispersion A, thus giving deodorizing fiber J.

### Example 128

The procedure of Example 118 was carried out in the same way except that deodorant dispersion K was used instead of deodorant dispersion A, thus giving deodorizing fiber K.

### Example 129

The procedure of Example 118 was carried out in the same way except that deodorant dispersion L was used instead of deodorant dispersion A, thus giving deodorizing fiber L.

### Example 130

The procedure of Example 118 was carried out in the same way except that deodorant dispersion M was used instead of deodorant dispersion A, thus giving deodorizing fiber M.

### Example 131

The procedure of Example 118 was carried out in the same way except that deodorant dispersion N was used instead of deodorant dispersion A, thus giving deodorizing fiber N.

### Example 132

The procedure of Example 118 was carried out in the same way except that deodorant dispersion O was used instead of deodorant dispersion A, thus giving deodorizing fiber O.

### Example 133

The procedure of Example 118 was carried out in the same way except that deodorant dispersion P was used instead of deodorant dispersion A, thus giving deodorizing fiber P.

### Example 134

The procedure of Example 118 was carried out in the same way except that deodorant dispersion B (p1) was used instead of deodorant dispersion A, thus giving deodorizing fiber B (p1).

### Example 135

The procedure of Example 118 was carried out in the same way except that deodorant dispersion F (p1) was used instead of deodorant dispersion A, thus giving deodorizing fiber F (p1).

### Example 136

The procedure of Example 118 was carried out in the same way except that deodorant dispersion J (p1) was used instead of deodorant dispersion A, thus giving deodorizing fiber J (p1).

### Example 137

The procedure of Example 118 was carried out in the same way except that deodorant dispersion B (p2) was used instead of deodorant dispersion A, thus giving deodorizing fiber B (p2).

### Example 138

The procedure of Example 118 was carried out in the same way except that deodorant dispersion F (p2) was used instead of deodorant dispersion A, thus giving deodorizing fiber F (p2).

### Example 139

The procedure of Example 118 was carried out in the same way except that deodorant dispersion J (p2) was used instead of deodorant dispersion A, thus giving deodorizing fiber J (p2).

### Example 140

The procedure of Example 118 was carried out in the same way except that deodorant composition dispersion A was used instead of deodorant dispersion A, thus giving deodorizing fiber A (mix).

### Example 141

The procedure of Example 118 was carried out in the same way except that deodorant composition dispersion B was used instead of deodorant dispersion A, thus giving deodorizing fiber B (mix).

### Example 142

The procedure of Example 118 was carried out in the same way except that deodorant composition dispersion C was used instead of deodorant dispersion A, thus giving deodorizing fiber C (mix).

### Example 143

The procedure of Example 118 was carried out in the same way except that deodorant composition dispersion D was used instead of deodorant dispersion A, thus giving deodorizing fiber D (mix).

### Example 144

The procedure of Example 118 was carried out in the same way except that deodorant dispersion A1 was used instead of deodorant dispersion A, thus giving deodorizing fiber A1.

### Example 145

The procedure of Example 118 was carried out in the same way except that deodorant dispersion A2 was used instead of deodorant dispersion A, thus giving deodorizing fiber A2.

### Example 146

The procedure of Example 118 was carried out in the same way except that deodorant dispersion A3 was used instead of deodorant dispersion A, thus giving deodorizing fiber A3.

### Comparative Example 38

The procedure of Example 118 was carried out in the same way except that 3 parts by weight of sample a was used instead of deodorant dispersion A, thus giving comparative fiber a (the content of the sample was 1.5 parts relative to 100 parts by weight of the fiber).

### Comparative Example 39

The procedure of Example 118 was carried out in the same way except that 3 parts by weight of sample composition a was used instead of deodorant dispersion A, thus giving comparative fiber aa (the content of the sample composition was 1.5 parts relative to 100 parts by weight of the fiber).

### Example 147

### Measurement of Deodorizing Effect of Deodorizing Fiber

20 g of deodorizing fiber A was placed in a Tedlar bag, 1 L of malodorous gas (containing 40 ppm of acetaldehyde gas, 40 ppm of ammonia gas, 10 ppm of hydrogen sulfide gas, and 40 ppm of acetic acid gas) was injected thereinto, and the bag was left to stand at room temperature. After 2 hours, the concentrations of gases remaining in the Tedlar bag were measured.

The other deodorizing fibers and comparative fibers were subjected to the same procedure, thus measuring the concentrations of gases remaining.

The results are given in Table 10. ND in the table denotes that evaluation was not carried out. The same applies below.

### Example 148

### Surface Condition of Deodorizing Fiber

The surface condition of the fiber to which the deodorant had been added was visually examined, and the results are given in Table 10.

TABLE 10

| | | | | | Surface |
|---|---|---|---|---|---|
| | Results of deodorizing effect | | | | condition |
| | Ammo- | Acetal- | Hydrogen | Acetic | obser- |
| | nia | dehyde | sulfide | acid | vation |
| Deodorizing fiber A | ND | 4 | ND | ND | Good |
| Deodorizing fiber B | ND | 4 | ND | ND | Good |
| Deodorizing fiber C | ND | 3 | ND | ND | Good |
| Deodorizing fiber D | ND | 2 | ND | ND | Good |
| Deodorizing fiber E | ND | 6 | ND | ND | Good |
| Deodorizing fiber F | ND | 6 | ND | ND | Good |
| Deodorizing fiber G | ND | 5 | ND | ND | Good |
| Deodorizing fiber H | ND | 5 | ND | ND | Good |
| Deodorizing fiber I | ND | 4 | ND | ND | Good |
| Deodorizing fiber J | ND | 4 | ND | ND | Good |
| Deodorizing fiber K | ND | 3 | ND | ND | Good |
| Deodorizing fiber L | ND | 2 | ND | ND | Good |
| Deodorizing fiber M | ND | 10 | ND | ND | Good |
| Deodorizing fiber N | ND | 10 | ND | ND | Good |
| Deodorizing fiber O | ND | 8 | ND | ND | Good |
| Deodorizing fiber P | ND | 8 | ND | ND | Good |
| Deodorizing fiber B | <0.2 | 4 | ND | ND | Good |
| (p1) | | | | | |
| Deodorizing fiber F | ND | 6 | ND | ND | Good |
| (p1) | | | | | |
| Deodorizing fiber J | ND | 4 | ND | ND | Good |
| (p1) | | | | | |
| Deodorizing fiber B | <0.2 | 4 | ND | ND | Good |
| (p2) | | | | | |
| Deodorizing fiber F | ND | 6 | ND | ND | Good |
| (p2) | | | | | |
| Deodorizing fiber J | ND | 4 | ND | ND | Good |
| (p2) | | | | | |
| Deodorizing fiber A | <0.2 | 10 | 3 | <1 | Good |
| (mix) | | | | | |
| Deodorizing fiber B | <0.2 | 10 | 3 | <1 | Good |
| (mix) | | | | | |
| Deodorizing fiber C | <0.2 | 8 | 3 | <1 | Good |
| (mix) | | | | | |
| Deodorizing fiber D | <0.2 | 8 | 3 | <1 | Good |
| (mix) | | | | | |
| Deodorizing fiber | ND | 8 | ND | ND | Some |
| A1 | | | | | partic- |
| | | | | | ulates |
| Deodorizing fiber | ND | 8 | ND | ND | Some |
| A2 | | | | | partic- |
| | | | | | ulates |
| Deodorizing fiber | ND | 8 | ND | ND | Some |
| A3 | | | | | partic- |
| | | | | | ulates |
| Comparative fiber a | ND | 18 | ND | ND | Good |
| Comparative fiber | 0.4 | 20 | 6 | 3 | Good |
| aa | | | | | |

From the above results, the fiber to which the aldehyde gas deodorant of the present invention has been added exhibits an excellent deodorizing effect for acetaldehyde. Furthermore, the fiber to which the deodorant composition of the present invention has been added exhibits an excellent deodorizing effect for bad odors due to acetaldehyde, ammonia, hydrogen sulfide, acetic acid, etc. Moreover, a good surface condition can be obtained when the fiber is coated with a dispersion in which the aldehyde gas deodorant of the present invention is dispersed, and an excellent deodorizing effect can be exhibited.

### Example 149

### Preparation of Deodorizing Carpet

A suspension was prepared by adding 2.1 parts by weight of deodorant A and 2.1 parts by weight of KB-3000 urethane-based binder (manufactured by Toagosei Co., Ltd.) to 100 parts by weight of purified water.

The carpet employed a polypropylene woven fabric as a carpet substrate, and polyester fiber pile threads given a backing treatment with a backing treatment liquid (200 parts of calcium carbonate, 3 parts of Emulgen 708 (manufactured by Kao Corporation), and 10 parts of water added to 100 parts of SBR latex, and the mixture subjected to mechanical foaming) at a weight per unit area of 700 g/m².

The surface fiber section of this carpet was coated with the suspension at 50 g/m², and dried naturally by allowing it to stand in a room for 3 hours, thus giving deodorizing carpet A (the amount of deodorant attached was 1 g/m² as a solids content, and the urethane binder was 0.3 g/m² as a solids content).

### Example 150

The procedure of Example 149 was carried out in the same way except that deodorant B was used instead of deodorant A, thus giving deodorizing carpet B.

### Example 151

The procedure of Example 149 was carried out in the same way except that deodorant C was used instead of deodorant A, thus giving deodorizing carpet C.

### Example 152

The procedure of Example 149 was carried out in the same way except that deodorant D was used instead of deodorant A, thus giving deodorizing carpet D.

### Example 153

The procedure of Example 149 was carried out in the same way except that deodorant E was used instead of deodorant A, thus giving deodorizing carpet E.

### Example 154

The procedure of Example 149 was carried out in the same way except that deodorant F was used instead of deodorant A, thus giving deodorizing carpet F.

### Example 155

The procedure of Example 149 was carried out in the same way except that deodorant G was used instead of deodorant A, thus giving deodorizing carpet G.

### Example 156

The procedure of Example 149 was carried out in the same way except that deodorant H was used instead of deodorant A, thus giving deodorizing carpet H.

### Example 157

The procedure of Example 149 was carried out in the same way except that deodorant I was used instead of deodorant A, thus giving deodorizing carpet I.

### Example 158

The procedure of Example 149 was carried out in the same way except that deodorant J was used instead of deodorant A, thus giving deodorizing carpet J.

### Example 159

The procedure of Example 149 was carried out in the same way except that deodorant K was used instead of deodorant A, thus giving deodorizing carpet K.

### Example 160

The procedure of Example 149 was carried out in the same way except that deodorant L was used instead of deodorant A, thus giving deodorizing carpet L.

### Example 161

The procedure of Example 149 was carried out in the same way except that deodorant M was used instead of deodorant A, thus giving deodorizing carpet M.

### Example 162

The procedure of Example 149 was carried out in the same way except that deodorant N was used instead of deodorant A, thus giving deodorizing carpet N.

### Example 163

The procedure of Example 149 was carried out in the same way except that deodorant O was used instead of deodorant A, thus giving deodorizing carpet O.

### Example 164

The procedure of Example 149 was carried out in the same way except that deodorant P was used instead of deodorant A, thus giving deodorizing carpet P.

### Comparative Example 40

The procedure of Example 149 was carried out in the same way except that sample a was used instead of deodorant A, thus giving comparative carpet a.

### Comparative Example 41

A solution was prepared by dissolving 2.1 parts by weight of adipic acid dihydrazide in 100 parts by weight of purified water. The surface fiber section of a carpet of the same type as that used in Example 149 was coated with this solution at 50 g/m², and dried naturally by allowing it to stand in a room for 3 hours, thus giving comparative carpet 2b.

### Comparative Example 42

The procedure of Comparative Example 41 was carried out in the same way except that purified water was used instead of the adipic acid dihydrazide aqueous solution, thus giving comparative carpet 2c (deodorant not added).

### Example 165

### Measurement of Aldehyde Deodorizing Performance

A 200 mm×200 mm test piece was cut out from deodorizing carpet A. This test piece was sealed in a Tedlar bag, 3 L of aldehyde test gas (containing 20 ppm of acetaldehyde gas and 20 ppm of formaldehyde) was injected thereinto, and the bag was left to stand at room temperature. After 2 hours, the concentrations of gases remaining in the Tedlar bag were measured.

The other deodorizing carpets and comparative carpets were subjected to the same procedure, thus measuring the concentrations of gases remaining.

The results are given in Table 11.

TABLE 11

| | Formaldehyde | Acetaldehyde |
|---|---|---|
| Deodorizing carpet A | <1 | 3 |
| Deodorizing carpet B | <1 | 3 |
| Deodorizing carpet C | <1 | 1 |
| Deodorizing carpet D | <1 | 1 |
| Deodorizing carpet E | <1 | 5 |
| Deodorizing carpet F | <1 | 5 |
| Deodorizing carpet G | <1 | 3 |
| Deodorizing carpet H | <1 | 2 |
| Deodorizing carpet I | <1 | 4 |
| Deodorizing carpet J | <1 | 4 |
| Deodorizing carpet K | <1 | 2 |
| Deodorizing carpet L | <1 | 2 |
| Deodorizing carpet M | <1 | 5 |
| Deodorizing carpet N | <1 | 5 |
| Deodorizing carpet O | <1 | 2 |
| Deodorizing carpet P | <1 | 2 |
| Comparative carpet a | 4 | 10 |
| Comparative carpet 2b | 2 | 8 |
| Comparative carpet 2c | 17 | 20 |

It can be seen that carpets to which the aldehyde gas deodorant of the present invention has been added have a high deodorizing effect for formaldehyde and acetaldehyde and exhibit an excellent aldehyde deodorizing effect compared with the Comparative Examples.

### Example 166

### Measurement of Amount of Aldehyde Diffusing

A 200 mm×200 mm test piece was cut out from deodorizing carpet A. This test piece was sealed in a Tedlar bag, and 4 L of nitrogen gas was further injected thereinto. This Tedlar bag was heated at 65° C. for 2 hours, and aldehyde gas in the Tedlar bag was collected by a DNPH cartridge (manufactured by SUPELCO). This DNPH cartridge was extracted with acetonitrile, formaldehyde and acetaldehyde in the extract were analyzed by high performance liquid chromatography (L-6000, manufactured by Hitachi, Ltd.), and the amounts of aldehydes diffusing per test piece (μg/test piece) were calculated.

The other deodorizing carpets and comparative carpets were subjected to the same procedure, and the amounts of aldehydes diffusing were calculated.

The results are given in Table 12.

Analytical conditions for high performance liquid chromatography
Eluent: acetonitrile/distilled water=50/50 (ratio by volume)
Column: ODS-80A (manufactured by GL Sciences Inc.)
Column temperature: 40° C., detector wavelength 360 nm

TABLE 12

| | Amount of | Amount of |
|---|---|---|
| | formaldehyde diffusing | acetaldehyde diffusing |
| | (μg/test piece) | (μg/test piece) |
| Deodorizing carpet A | <0.1 | 0.3 |
| Deodorizing carpet B | <0.1 | 0.3 |
| Deodorizing carpet C | <0.1 | 0.1 |
| Deodorizing carpet D | <0.1 | 0.1 |
| Deodorizing carpet E | <0.1 | 0.5 |
| Deodorizing carpet F | <0.1 | 0.5 |
| Deodorizing carpet G | <0.1 | 0.3 |
| Deodorizing carpet H | <0.1 | 0.3 |
| Deodorizing carpet I | <0.1 | 0.2 |
| Deodorizing carpet J | <0.1 | 0.2 |
| Deodorizing carpet K | <0.1 | 0.1 |
| Deodorizing carpet L | <0.1 | 0.1 |
| Deodorizing carpet M | <0.1 | 0.5 |
| Deodorizing carpet N | <0.1 | 0.5 |
| Deodorizing carpet O | <0.1 | 0.3 |
| Deodorizing carpet P | <0.1 | 0.3 |
| Comparative carpet a | 0.2 | 2.2 |
| Comparative carpet 2b | 0.1 | 1.7 |
| Comparative carpet 2c | 1.0 | 3.8 |

It can be seen that carpets to which the aldehyde gas deodorant of the present invention has been added suppress the amounts of formaldehyde and acetaldehyde diffusing compared with the Comparative Examples. This suggests that the carpet of the present invention has an excellent effect in suppressing aldehyde volatilization.

### Example 167

### Preparation of Deodorizing Steel Sheet

A paste composition was obtained by adding 70 parts by weight of an acrylic resin (J-500, manufactured by S C Johnson Polymer), 3 parts by weight of a dispersant (BYK-110 manufactured by BYK Chemie), 2 parts by weight of a thickener (Benton SD2, manufactured by Wilbur-Ellis Co.), and 200 parts by weight of deodorant A to 100 parts by weight of xylene, and kneading and dispersing well by means of a three roll mixer. This was diluted by 10 times with xylene, and opposite sides of a 70×150 mm zinc plated steel sheet were coated therewith at a coating thickness of 100 μm and air-dried overnight, thus giving deodorizing steel sheet A1.

### Example 168

The procedure of Example 167 was carried out in the same way except that deodorant composition A was used instead of deodorant A, thus giving deodorizing steel sheet A2.

### Comparative Example 43

The procedure of Example 167 was carried out in the same way except that sample a was used instead of deodorant A, thus giving comparative steel sheet a1.

### Comparative Example 44

The procedure of Example 167 was carried out in the same way except that sample composition a was used instead of deodorant A, thus giving comparative steel sheet a2.

### Example 169

### Measurement of Deodorizing Effect for Deodorizing Steel Sheet

One sheet of deodorizing steel sheet A1 was placed in a Tedlar bag, 1 L of malodorous gas (containing 20 ppm of acetaldehyde gas, 40 ppm of ammonia gas, 10 ppm of hydrogen sulfide gas, and 40 ppm of acetic acid gas) was injected thereinto, and the bag was left to stand at room temperature. After 2 hours, the concentrations of gases remaining in the Tedlar bag were measured.

The other deodorizing steel sheets and comparative steel sheets were subjected to the same procedure, thus measuring the concentrations of gases remaining.

The results are given in Table 13.

TABLE 13

| | Ammo- | Acetal- | Hydrogen | Acetic |
|---|---|---|---|---|
| | nia | dehyde | sulfide | acid |
| Deodorizing steel sheet A1 | ND | 8 | ND | ND |
| Deodorizing steel sheet A2 | 2 | 10 | 6 | 2 |
| Comparative steel sheet a1 | ND | 19 | ND | ND |
| Comparative steel sheet a2 | 12 | 20 | 8 | 5 |

It can be seen from the results in Table 13 that steel sheets to which the aldehyde gas deodorant of the present invention has been added exhibit an excellent deodorizing effect for acetaldehyde. Furthermore, steel sheets to which the deodorant composition of the present invention has been added exhibit an excellent deodorizing effect for bad odors due to acetaldehyde, ammonia, hydrogen sulfide, acetic acid, etc.

### Example 170

### Nozzle Liquid Passage Characteristics

A suspension was prepared by adding 27 parts by weight of deodorant dispersion B and 8 parts by weight of a urethane-based binder (KB-3000, manufactured by Toagosei Co., Ltd.) to 100 parts by weight of purified water. 200 g of this suspension was placed in a stainless container, pressure was applied thereto so that the internal pressure was 0.1 MPa, and the suspension was sprayed via one fluid nozzle (Unijet TG Full Cone, flow rate size 0.3, manufactured by Spraying Systems Co., Japan). After the entire amount was sprayed, 200 g of the suspension was again placed in the container, and spraying was carried out in the same manner. This procedure was repeated a total of five times, and the spraying time was measured for each trial. The other deodorizing dispersions were subjected to the same procedure, and the spraying times were measured. The results are given in Table 14.

TABLE 14

| | Spraying time (sec) | | | | |
|---|---|---|---|---|---|
| | 1st | 2nd | 3rd | 4th | 5th |
| | time | time | time | time | time |
| Deodorant dispersion B | 160 | 170 | 190 | 230 | 440 |
| Deodorant dispersion F | 160 | 170 | 190 | 230 | 440 |
| Deodorant dispersion J | 160 | 170 | 190 | 230 | 440 |
| Deodorant dispersion B (p1) | 160 | 160 | 160 | 160 | 160 |
| Deodorant dispersion F (p1) | 160 | 160 | 160 | 160 | 160 |
| Deodorant dispersion J (p1) | 160 | 160 | 160 | 160 | 160 |
| Deodorant dispersion B (p2) | 160 | 165 | 175 | 180 | 195 |
| Deodorant dispersion F (p2) | 160 | 165 | 175 | 180 | 195 |
| Deodorant dispersion J (p2) | 160 | 165 | 175 | 180 | 195 |

It can be seen from the above results that when the deodorant dispersion of the present invention is sprayed via a nozzle, superior nozzle liquid passage characteristics are exhibited when a humectant is added.

### Example 171

### Preparation of Deodorizing Dispersion As

While stirring 100 parts by weight of an acidic silica sol (aqueous, SNOWTEX O, manufactured by Nissan Chemical Industries, Ltd., silica content: 20%, measured pH 2.7) at room temperature, 1 part by weight of aminoguanidine hydrochloride was added and dissolved therein, thus giving deodorizing dispersion As. The aminoguanidine hydrochloride was 5 parts by weight relative to 100 parts by weight of the silica content in deodorizing dispersion As.

### Example 172

### Preparation of Deodorizing Dispersion Bs

The same procedure as for the preparation of deodorizing dispersion As was carried out except that aminoguanidine sulfate was used instead of aminoguanidine hydrochloride, thus giving deodorizing dispersion Bs. The aminoguanidine sulfate was 5 parts by weight relative to 100 parts by weight of the silica content in deodorant dispersion Bs.

### Example 173

### Preparation of Deodorizing Dispersion Cs

The same procedure as for the preparation of deodorizing dispersion As was carried out except that diaminoguanidine hydrochloride was used instead of aminoguanidine hydrochloride, thus giving deodorizing dispersion Cs. The diaminoguanidine hydrochloride was 5 parts by weight relative to 100 parts by weight of the silica content in deodorizing dispersion Cs.

### Example 174

### Preparation of Deodorizing Dispersion Ds

The same procedure as for the preparation of deodorizing dispersion As was carried out except that triaminoguanidine hydrochloride was used instead of aminoguanidine hydrochloride, thus giving deodorizing dispersion Ds. The triaminoguanidine hydrochloride was 5 parts by weight relative to 100 parts by weight of the silica content in deodorant dispersion Ds.

### Example 175

### Preparation of Deodorizing Dispersion Es

The same procedure as for the preparation of deodorizing dispersion As was carried out except that 5 parts by weight of aminoguanidine hydrochloride was added, thus giving deodorizing dispersion Es. The aminoguanidine hydrochloride was 25 parts by weight relative to 100 parts by weight of the silica content in deodorizing dispersion Es.

### Example 176

### Preparation of Deodorizing Dispersion Fs

The same procedure as for the preparation of deodorizing dispersion Bs was carried out except that 5 parts by weight of aminoguanidine sulfate was added, thus giving deodorizing dispersion Fs. The aminoguanidine sulfate was 25 parts by weight relative to 100 parts by weight of the silica content in deodorizing dispersion Fs.

### Example 177

### Preparation of Deodorizing Dispersion Gs

The same procedure as for the preparation of deodorizing dispersion Cs was carried out except that 5 parts by weight of diaminoguanidine hydrochloride was added, thus giving deodorizing dispersion Gs. The diaminoguanidine hydrochloride was 25 parts by weight relative to 100 parts by weight of the silica content in deodorizing dispersion Gs.

### Example 178

### Preparation of Deodorizing Dispersion Hs

The same procedure as for the preparation of deodorizing dispersion Ds was carried out except that 5 parts by weight of triaminoguanidine hydrochloride was added, thus giving deodorizing dispersion Hs. The triaminoguanidine hydrochloride was 25 parts by weight relative to 100 parts by weight of the silica content in deodorizing dispersion Hs.

### Comparative Example 45

### Preparation of Comparative Sample as

The same procedure as for the preparation of deodorizing dispersion As was carried out except that 50 parts by weight of aminoguanidine hydrochloride was added, thus giving sample as. The aminoguanidine hydrochloride was 250 parts by weight relative to 100 parts by weight of the silica content in sample as.

### Comparative Example 46

### Preparation of Comparative Sample bs

The same procedure as for the preparation of deodorizing dispersion Bs was carried out except that 50 parts by weight of aminoguanidine sulfate was added, thus giving sample bs. The aminoguanidine sulfate was 250 parts by weight relative to 100 parts by weight of the silica content in sample bs. The full amount of the aminoguanidine sulfate did not dissolve, but the sample was used as it was.

### Comparative Example 47

### Preparation of Comparative Sample cs

The same procedure as for the preparation of deodorizing dispersion Cs was carried out except that 50 parts by weight of diaminoguanidine hydrochloride was added, thus giving sample cs. The diaminoguanidine hydrochloride was 250 parts by weight relative to 100 parts by weight of the silica content in sample cs.

### Comparative Example 48

### Preparation of Comparative Sample ds

The same procedure as for the preparation of deodorizing dispersion Ds was carried out except that 50 parts by weight of triaminoguanidine hydrochloride was added, thus giving sample ds. The triaminoguanidine hydrochloride was 250 parts by weight relative to 100 parts by weight of the silica content in sample ds.

### Comparative Example 49

### Preparation of Comparative Sample es

The same procedure as for the preparation of deodorizing dispersion Es was carried out except that adipic acid dihydrazide was used instead of aminoguanidine hydrochloride, thus giving sample es. The adipic acid dihydrazide was 25 parts by weight relative to 100 parts by weight of the silica content in sample es.

### Comparative Example 50

### Preparation of Comparative Sample fs

The same procedure as for the preparation of deodorizing dispersion As was carried out except that urea was used instead of aminoguanidine hydrochloride, thus giving sample fs. The urea was 5 parts by weight relative to 100 parts by weight of the silica content in sample fs.

### Comparative Example 51

### Preparation of Comparative Sample gs

The same procedure as for the preparation of deodorizing dispersion Es was carried out except that urea was used instead of aminoguanidine hydrochloride, thus giving sample gs. The urea was 25 parts by weight relative to 100 parts by weight of the silica content in sample gs.

### Comparative Example 52

### Preparation of Comparative Sample hs

The same procedure as for the preparation of deodorizing dispersion As was carried out except that an ammonia-stabilized silica sol (SNOWTEX N, pH 9.5, manufactured by Nissan Chemical Industries, Ltd.) was used instead of SNOWTEX O, thus giving sample hs. However, this sample hs gelled and did not function as a dispersion.

### Example 179

### Preparation of Deodorizing Board As

One side of a 9 mm thick particleboard was coated with deodorizing dispersion As in an amount of 29 g/m², and dried naturally for 1 hour, thus giving deodorizing board As. One side of deodorizing board As was coated with silica at 5.7 g/m² and aminoguanidine hydrochloride at 0.3 g/m².

### Example 180

### Preparation of Deodorizing Board Bs

The same procedure as for the preparation of deodorizing board As was carried out except that deodorizing dispersion Bs was used instead of deodorizing dispersion As, thus giving deodorizing board Bs. One side of deodorizing board Bs was coated with silica at 5.7 g/m² and aminoguanidine sulfate at 0.3 g/m².

### Example 181

### Preparation of Deodorizing Board Cs

The same procedure as for the preparation of deodorizing board As was carried out except that deodorizing dispersion Cs was used instead of deodorizing dispersion As, thus giving deodorizing board Cs. One side of deodorizing board Cs was coated with silica at 5.7 g/m² and diaminoguanidine hydrochloride at 0.3 g/m².

### Example 182

### Preparation of Deodorizing Board Ds

The same procedure as for the preparation of deodorizing board As was carried out except that deodorizing dispersion Ds was used instead of deodorizing dispersion As, thus giving deodorizing board Ds. One side of deodorizing board Ds was coated with silica at 5.7 g/m² and triaminoguanidine hydrochloride at 0.3 g/m².

### Example 183

### Preparation of Deodorizing Board Es

The same procedure as for the preparation of deodorizing board As was carried out except that deodorizing dispersion Es was applied at 25 g/m² instead of deodorizing dispersion As at 29 g/m², thus giving deodorizing board Es. One side of deodorizing board Es was coated with silica at 4.8 g/m² and aminoguanidine hydrochloride at 1.2 g/m².

### Example 184

### Preparation of Deodorizing Board Fs

The same procedure as for the preparation of deodorizing board Es was carried out except that deodorizing dispersion Fs was used instead of deodorizing dispersion Es, thus giving deodorizing board Fs. One side of deodorizing board Fs was coated with silica at 4.8 g/m² and aminoguanidine sulfate at 1.2 g/m².

### Example 185

### Preparation of Deodorizing Board Gs

The same procedure as for the preparation of deodorizing board Es was carried out except that deodorizing dispersion Gs was used instead of deodorizing dispersion Es, thus giving deodorizing board Gs. One side of deodorizing board Gs was coated with silica at 4.8 g/m² and diaminoguanidine hydrochloride at 1.2 g/m².

### Example 186

### Preparation of Deodorizing Board Hs

The same procedure as for the preparation of deodorizing board Es was carried out except that deodorizing dispersion Hs was used instead of deodorizing dispersion Es, thus giving deodorizing board Hs. One side of deodorizing board Hs was coated with silica at 4.8 g/m² and triaminoguanidine hydrochloride at 1.2 g/m².

### Comparative Example 53

### Preparation of Sample Board as

The same procedure as for the preparation of deodorizing board As was carried out except that sample as was applied at 13 g/m² instead of deodorizing dispersion As at 29 g/m², thus preparing sample board as. One side of sample board as was coated with silica at 1.0 g/m² and aminoguanidine hydrochloride at 5.0 g/m².

### Comparative Example 54

### Preparation of Sample Board bs

The same procedure as for the preparation of sample board as was carried out except that sample bs was used instead of sample as, thus preparing sample board bs. One side of sample board as was coated with silica at 1.0 g/m² and aminoguanidine sulfate at 5.0 g/m².

### Comparative Example 55

### Preparation of Sample Board cs

The same procedure as for the preparation of sample board as was carried out except that sample cs was used instead of sample as, thus preparing sample board cs. One side of sample board cs was coated with silica at 1.0 g/m² and diaminoguanidine hydrochloride at 5.0 g/m².

### Comparative Example 56

### Preparation of Sample Board ds

The same procedure as for the preparation of sample board as was carried out except that sample ds was used instead of sample as, thus preparing sample board ds. One side of sample board ds was coated with silica at 1.0 g/m² and triaminoguanidine hydrochloride at 5.0 g/m².

### Comparative Example 57

### Preparation of Sample Board es

The same procedure as for the preparation of deodorizing board Es was carried out except that sample es was used instead of deodorizing dispersion Es, thus preparing sample board es. One side of sample board es was coated with silica at 4.8 g/m² and adipic acid dihydrazide at 1.2 g/m².

### Comparative Example 58

### Preparation of Sample Board fs

The same procedure as for the preparation of deodorizing board As was carried out except that sample fs was used instead of deodorizing dispersion As, thus preparing sample board fs. One side of sample board fs was coated with silica at 5.7 g/m² and urea at 0.3 g/m².

### Comparative Example 59

### Preparation of Sample Board gs

The same procedure as for the preparation of deodorizing board Es was carried out except that sample gs was used instead of deodorizing dispersion Es, thus preparing sample board gs. One side of sample board gs was coated with silica at 4.8 g/m² and urea at 1.2 g/m².

### Comparative Example 60

### Preparation of Sample Board is

One side of a particleboard was coated with SNOWTEX 0 at 30 g/m², and dried naturally for 1 hour, thus giving sample board is. One side of sample board is was coated with silica at 6.0 g/m².

### Comparative Example 61

### Preparation of Sample Board js

The same procedure as for the preparation of sample board is was carried out except that a 20 wt % aminoguanidine hydrochloride aqueous solution was used instead of SNOWTEX O, thus preparing sample board js. One side of sample board js was coated with aminoguanidine hydrochloride at 6.0 g/m².

### Comparative Example 62

### Preparation of Sample Board ks

The same procedure as for the preparation of sample board is was carried out except that a 20 wt % aminoguanidine sulfate aqueous solution was used instead of SNOWTEX O, thus preparing sample board ks. One side of sample board ks was coated with aminoguanidine sulfate at 6.0 g/m².

### Comparative Example 63

### Preparation of Sample Board ls

The same procedure as for the preparation of sample board is was carried out except that a 20 wt % diaminoguanidine hydrochloride aqueous solution was used instead of SNOWTEX O, thus preparing sample board ls. One side of sample board ls was coated with diaminoguanidine hydrochloride at 6.0 g/m².

### Comparative Example 64

### Preparation of Sample Board ms

The same procedure as for the preparation of sample board is was carried out except that a 20 wt % triaminoguanidine hydrochloride aqueous solution was used instead of SNOWTEX O, thus preparing sample board ms. One side of sample board ms was coated with triaminoguanidine hydrochloride at 6.0 g/m².

### Comparative Example 65

### Preparation of Sample Board ns

The same procedure as for the preparation of sample board is was carried out except that purified water was used instead of SNOWTEX O, thus preparing sample board ns.

### Example 187

Measurement of Amount of Aldehyde Diffusing from Deodorizing Particleboard, Etc.

A 10 cm long×8 cm wide test piece was cut out from deodorizing board As. This test piece was sealed in a Tedlar bag, and 4 L of nitrogen gas was further injected thereinto. This Tedlar bag was heated at 65° C. for 2 hours, and aldehyde gas in the Tedlar bag was collected by a DNPH cartridge (manufactured by SUPELCO). This DNPH cartridge was extracted with acetonitrile, formaldehyde and acetaldehyde in the extract were analyzed by high performance liquid chromatography (L-6000, manufactured by Hitachi, Ltd.), and the amounts of aldehydes diffusing per test piece (μg/test piece) were calculated. The other deodorizing boards and comparative sample boards were subjected to the same procedure, and the amounts of aldehydes diffusing were calculated.

The results are given in Table 15.

Analytical conditions for high performance liquid chromatography
Eluent: acetonitrile/distilled water=50/50 (ratio by volume)
Column: ODS-80A (manufactured by GL Sciences Inc.)
Column temperature: 40° C., detector wavelength 360 nm

TABLE 15

| | Amount of | Amount of |
|---|---|---|
| | formaldehyde diffusing | acetaldehyde diffusing |
| | (μg/test piece) | (μg/test piece) |
| Deodorizing board As | 0.2 | 0.3 |
| Deodorizing board Bs | 0.2 | 0.3 |
| Deodorizing board Cs | 0.2 | 0.2 |
| Deodorizing board Ds | 0.2 | 0.2 |
| Deodorizing board Es | 0.2 | 0.8 |
| Deodorizing board Fs | 0.2 | 0.8 |
| Deodorizing board Gs | 0.2 | 0.6 |
| Deodorizing board Hs | 0.2 | 0.6 |
| Sample board as | 1.0 | 2.2 |
| Sample board bs | 1.0 | 2.2 |
| Sample board cs | 0.8 | 2.0 |
| Sample board ds | 0.8 | 2.0 |
| Sample board es | 0.8 | 2.5 |
| Sample board fs | 1.6 | 5.6 |
| Sample board gs | 1.6 | 5.6 |
| Sample board is | 2.0 | 5.8 |
| Sample board js | 0.8 | 2.8 |
| Sample board ks | 0.8 | 2.8 |
| Sample board ls | 0.6 | 2.8 |
| Sample board ms | 0.6 | 2.6 |
| Sample board ns | 2.0 | 5.8 |

It can be seen that the particleboards treated with the deodorant of the present invention suppress the amounts of formaldehyde and acetaldehyde diffusing compared with the Comparative Examples. This suggests that the particleboard of the present invention has an excellent effect in suppressing aldehyde volatilization.

### Example 188

### Preparation of Deodorizing Polyurethane Foam As

One side of a sheet formed by molding regenerated polyurethane foam chips at a thickness of 15 mm using an adhesive was coated with deodorizing dispersion As at 29 g/m², and dried naturally by allowing it to stand in a room for 1 hour, thus giving deodorizing polyurethane foam As. One side of deodorizing polyurethane foam As was coated with silica at 5.7 g/m² and aminoguanidine hydrochloride at 0.3 g/m².

### Example 189

### Preparation of Deodorizing Polyurethane Foam Bs

The same procedure as for the preparation of deodorizing polyurethane foam As was carried out in the same way except that deodorizing dispersion Bs was used instead of deodorizing dispersion As, thus giving deodorizing polyurethane foam Bs. One side of deodorizing polyurethane foam Bs was coated with silica at 5.7 g/m² and aminoguanidine sulfate at 0.3 g/m².

### Example 190

### Preparation of Deodorizing Polyurethane Foam Cs

The same procedure as for the preparation of deodorizing polyurethane foam As was carried out except that deodorizing dispersion Cs was used instead of deodorizing dispersion As, thus giving deodorizing polyurethane foam Cs. One side of deodorizing polyurethane foam Cs was coated with silica at 5.7 g/m² and diaminoguanidine hydrochloride at 0.3 g/m².

### Example 191

### Preparation of Deodorizing Polyurethane Foam Ds

The same procedure as for the preparation of deodorizing polyurethane foam As was carried out except that deodorizing dispersion Ds was used instead of deodorizing dispersion As, thus giving deodorizing polyurethane foam Ds. One side of deodorizing polyurethane foam Ds was coated with silica at 5.7 g/m² and triaminoguanidine hydrochloride at 0.3 g/m².

### Example 192

### Preparation of Deodorizing Polyurethane Foam Es

The same procedure as for the preparation of deodorizing polyurethane foam As was carried out except that deodorizing dispersion Es was applied at 25 g/m² instead of deodorizing dispersion As at 29 g/m², thus giving deodorizing polyurethane foam Es. One side of deodorizing polyurethane foam Es was coated with silica at 4.8 g/m² and aminoguanidine hydrochloride at 1.2 g/m².

### Example 193

### Preparation of Deodorizing Polyurethane Foam Fs

The same procedure as for the preparation of deodorizing polyurethane foam Es was carried out except that deodorizing dispersion Fs was used instead of deodorizing dispersion Es, thus giving deodorizing polyurethane foam Fs. One side of deodorizing polyurethane foam Fs was coated with silica at 4.8 g/m² and aminoguanidine sulfate at 1.2 g/m².

### Example 194

### Preparation of Deodorizing Polyurethane Foam Gs

The same procedure as for the preparation of deodorizing polyurethane foam Es was carried out except that deodorizing dispersion Gs was used instead of deodorizing dispersion Es, thus giving deodorizing polyurethane foam Gs. One side of deodorizing polyurethane foam Gs was coated with silica at 4.8 g/m² and diaminoguanidine hydrochloride at 1.2 g/m².

### Example 195

### Preparation of Deodorizing Polyurethane Foam Hs

The same procedure as for the preparation of deodorizing polyurethane foam Es was carried out except that deodorizing dispersion Hs was used instead of deodorizing dispersion Es, thus giving deodorizing polyurethane foam Hs. One side of deodorizing polyurethane foam Hs was coated with silica at 4.8 g/m² and triaminoguanidine hydrochloride at 1.2 g/m².

### Comparative Example 66

### Preparation of Sample Polyurethane Foam as

The same procedure as for the preparation of deodorizing polyurethane foam As was carried out except that sample as was applied at 13 g/m² instead of deodorizing dispersion As at 29 g/m², thus giving sample polyurethane foam as. One side of sample polyurethane foam as was coated with silica at 1.0 g/m² and aminoguanidine hydrochloride at 5.0 g/m².

### Comparative Example 67

### Preparation of Sample Polyurethane Foam bs

The same procedure as for the preparation of sample polyurethane foam as was carried out except that sample bs was used instead of sample as, thus giving sample polyurethane foam bs. One side of sample polyurethane foam bs was coated with silica at 1.0 g/m² and aminoguanidine sulfate at 5.0 g/m².

### Comparative Example 68

### Preparation of Sample Polyurethane Foam cs

The same procedure as for the preparation of sample polyurethane foam as was carried out except that sample cs was used instead of sample as, thus giving sample polyurethane foam cs. One side of sample polyurethane foam cs was coated with silica at 1.0 g/m² and diaminoguanidine hydrochloride at 5.0 g/m².

### Comparative Example 69

### Preparation of Sample Polyurethane Foam ds

The same procedure as for the preparation of sample polyurethane foam as was carried out except that sample ds was used instead of sample as, thus giving sample polyurethane foam ds. One side of sample polyurethane foam ds was coated with silica at 1.0 g/m² and triaminoguanidine hydrochloride at 5.0 g/m².

### Comparative Example 70

### Preparation of Sample Polyurethane Foam es

The same procedure as for the preparation of deodorizing polyurethane foam Es was carried out except that sample es was used instead of deodorizing dispersion Es, thus giving sample polyurethane foam es. One side of sample polyurethane foam es was coated with silica at 4.8 g/m² and adipic acid dihydrazide at 1.2 g/m².

### Comparative Example 71

### Preparation of Sample Polyurethane Foam fs

The same procedure as for the preparation of deodorizing polyurethane foam As was carried out except that sample fs was used instead of deodorizing dispersion As, thus giving sample polyurethane foam fs. One side of sample polyurethane foam fs was coated with silica at 5.7 g/m² and urea at 0.3 g/m².

### Comparative Example 72

### Preparation of Sample Polyurethane Foam gs

The same procedure as for the preparation of deodorizing polyurethane foam Es was carried out except that sample gs was used instead of deodorizing dispersion Es, thus giving sample polyurethane foam gs. One side of sample polyurethane foam gs was coated with silica at 4.8 g/m² and urea at 1.2 g/m².

### Comparative Example 73

### Preparation of Sample Polyurethane Foam is

One side of a sheet formed by molding regenerated polyurethane foam chips at a thickness of 15 mm using an adhesive was coated with SNOWTEX 0 at 30 g/m², and dried naturally by allowing it to stand for 1 hour, thus giving sample polyurethane foam is. One side of sample polyurethane foam is was coated with silica at 6.0 g/m².

### Comparative Example 74

### Preparation of Sample Polyurethane Foam js

The same procedure as for the preparation of sample polyurethane foam is was carried out except that a 20 wt % aminoguanidine hydrochloride aqueous solution was used instead of SNOWTEX O, thus giving sample polyurethane foam js. One side of sample polyurethane foam js was coated with aminoguanidine hydrochloride at 6.0 g/m².

### Comparative Example 75

### Preparation of Sample Polyurethane Foam ks

The same procedure as for the preparation of sample polyurethane foam is was carried out except that a 20 wt % aminoguanidine sulfate aqueous solution was used instead of SNOWTEX O, thus giving sample polyurethane foam ks. One side of sample polyurethane foam ks was coated with aminoguanidine sulfate at 6.0 g/m².

### Comparative Example 76

### Preparation of Sample Polyurethane Foam ls

The same procedure as for the preparation of sample polyurethane foam is was carried out except that a 20 wt % diaminoguanidine hydrochloride aqueous solution was used instead of SNOWTEX O, thus giving sample polyurethane foam ls. One side of sample polyurethane foam ls was coated with diaminoguanidine hydrochloride at 6.0 g/m².

### Comparative Example 77

### Preparation of Sample Polyurethane Foam ms

The same procedure as for the preparation of sample polyurethane foam is was carried out except that a 20 wt % triaminoguanidine hydrochloride aqueous solution was used instead of SNOWTEX O, thus giving sample polyurethane foam ms. One side of sample polyurethane foam ms was coated with triaminoguanidine hydrochloride at 6.0 g/m².

### Comparative Example 78

### Preparation of Sample Polyurethane Foam ns

The same procedure as for the preparation of sample polyurethane foam is was carried out except that purified water was used instead of SNOWTEX O, thus giving sample polyurethane foam ns.

### Example 196

Measurement of Amount of Aldehyde Diffusing from Deodorizing Polyurethane Foam, Etc.

A 10 cm long×8 cm wide test piece was cut out from deodorizing polyurethane foam As. This test piece was sealed in a Tedlar bag, and 4 L of nitrogen gas was further injected thereinto. This Tedlar bag was heated at 65° C. for 2 hours, and aldehyde gas in the Tedlar bag was collected by a DNPH cartridge (manufactured by SUPELCO). This DNPH cartridge was extracted with acetonitrile, formaldehyde and acetaldehyde in the extract were analyzed by high performance liquid chromatography (L-6000, manufactured by Hitachi, Ltd.) (the same analytical conditions as above were used), and the amounts of aldehydes diffusing per test piece (μg/test piece) were calculated. The other deodorizing polyurethane foams and comparative sample polyurethane foams were subjected to the same procedure, and amounts of aldehydes diffusing were calculated. The results are given in Table 16.

Analytical conditions for high performance liquid chromatography
Eluent: acetonitrile/distilled water=50/50 (ratio by volume)
Column: ODS-80A (manufactured by GL Science Inc.)
Column temperature: 40° C., detector wavelength 360 nm

TABLE 16

| | Amount of | Amount of |
|---|---|---|
| | formaldehyde diffusing | acetaldehyde diffusing |
| | (μg/test piece) | (μg/test piece) |
| Deodorizing | <0.1 | 0.2 |
| polyurethane foam As | | |
| Deodorizing | <0.1 | 0.2 |
| polyurethane foam Bs | | |
| Deodorizing | <0.1 | 0.2 |
| polyurethane foam Cs | | |
| Deodorizing | <0.1 | 0.2 |
| polyurethane foam Ds | | |
| Deodorizing | <0.1 | 0.2 |
| polyurethane foam Es | | |
| Deodorizing | <0.1 | 0.2 |
| polyurethane foam Fs | | |
| Deodorizing | <0.1 | 0.2 |
| polyurethane foam Gs | | |
| Deodorizing | <0.1 | 0.2 |
| polyurethane foam Hs | | |
| Sample polyurethane | <0.1 | 0.8 |
| foam as | | |
| Sample polyurethane | <0.1 | 0.8 |
| foam bs | | |
| Sample polyurethane | <0.1 | 0.7 |
| foam cs | | |
| Sample polyurethane | <0.1 | 0.7 |
| foam ds | | |
| Sample polyurethane | <0.1 | 0.9 |
| foam es | | |
| Sample polyurethane | 0.4 | 1.4 |
| foam fs | | |
| Sample polyurethane | 0.4 | 1.4 |
| foam gs | | |
| Sample polyurethane | 0.6 | 1.5 |
| foam is | | |
| Sample polyurethane | <0.1 | 0.8 |
| foam js | | |
| Sample polyurethane | <0.1 | 0.8 |
| foam ks | | |
| Sample polyurethane | <0.1 | 0.7 |
| foam ls | | |
| Sample polyurethane | <0.1 | 0.7 |
| foam ms | | |
| Sample polyurethane | 0.6 | 1.5 |
| foam ns | | |

### Example 197

### Preparation of Deodorizing Cloth As

Deodorizing dispersion As was diluted by 5.2 times with water, and this diluted liquid was sprayed on 100% cotton cloth in an amount of 50 g/m². Following this, the cloth was dried at 150° C., thus giving deodorizing cloth As. The deodorizing cloth As was coated with silica at 1.9 g/m² and aminoguanidine hydrochloride at 0.1 g/m².

### Example 198

### Preparation of Deodorizing Cloth Bs

The same procedure as for the preparation of deodorizing cloth As was carried out except that deodorizing dispersion Bs was used instead of deodorizing dispersion As, thus giving deodorizing cloth Bs. Deodorizing cloth Bs was coated with silica at 1.9 g/m² and aminoguanidine sulfate at 0.1 g/m².

### Example 199

### Preparation of Deodorizing Cloth Es

The same procedure as for the preparation of deodorizing cloth As was carried out except that a 6.0 times water dilution of deodorizing dispersion Es was used instead of the 5.2 times water dilution of deodorizing dispersion As, thus giving deodorizing cloth Es. Deodorizing cloth Es was coated with silica at 1.6 g/m² and aminoguanidine hydrochloride at 0.4 g/m².

### Example 200

### Preparation of Deodorizing Cloth Fs

The same procedure as for the preparation of deodorizing cloth Es was carried out except that deodorizing dispersion Fs was used instead of deodorizing dispersion Es, thus giving deodorizing cloth Fs. Deodorizing cloth Fs was coated with silica at 1.6 g/m² and aminoguanidine sulfate at 0.4 g/m².

### Comparative Example 79

### Preparation of Sample Cloth es

The same procedure as for the preparation of deodorizing cloth Es was carried out except that sample es was used instead of deodorizing dispersion Es, thus giving sample cloth es. Sample cloth es was coated with silica at 1.6 g/m² and adipic acid dihydrazide at 0.4 g/m².

### Comparative Example 80

### Preparation of Sample Cloth gs

The same procedure as for the preparation of deodorizing cloth Es was carried out except that sample gs was used instead of deodorizing dispersion Es, thus giving sample cloth gs. Sample cloth gs was coated with silica at 1.6 g/m² and urea at 0.4 g/m².

### Comparative Example 81

### Preparation of Sample Cloth js

The same procedure as for the preparation of deodorizing cloth As was carried out except that a 4 wt % aminoguanidine hydrochloride aqueous solution was used instead of the 5.2 times water dilution of deodorizing dispersion As, thus giving sample cloth js. Sample cloth js was coated with aminoguanidine hydrochloride at 2.0 g/m².

### Comparative Example 82

### Preparation of Sample Cloth ks

The same procedure as for the preparation of deodorizing cloth As was carried out except that a 4 wt % aminoguanidine sulfate aqueous solution was used instead of the 5.2 times water dilution of deodorizing dispersion As, thus giving sample cloth ks. Sample cloth ks was coated with aminoguanidine sulfate at 2.0 g/m².

### Example 201

### Measurement of Deodorizing Effect for Deodorizing Cloth, Etc.

A 10 cm×10 cm test piece was cut out from deodorizing cloth As. This test piece was placed in a Tedlar bag, 1 L of air containing 300 ppm of acetaldehyde gas was injected thereinto, and the bag was left to stand at room temperature. After 2 hours, the concentration of acetaldehyde gas remaining in the Tedlar bag was measured by a gas detector tube (manufactured by Gastec Corporation). The other deodorizing cloths and sample cloths were subjected to the same procedure, thus measuring the concentrations of gas remaining. The results are given in Table 17.

TABLE 17

| | Concentration of acetaldehyde remaining (ppm) |
|---|---|
| Deodorizing cloth As | 12 |
| Deodorizing cloth Bs | 12 |
| Deodorizing cloth Fs | 3 |
| Deodorizing cloth Gs | 3 |
| Sample cloth es | 70 |
| Sample cloth gs | 180 |
| Sample cloth js | 50 |
| Sample cloth ks | 40 |

From the results, the cloths treated with the deodorant of the present invention exhibit an excellent deodorizing effect for acetaldehyde.

### Example 202

### Preparation of Deodorizing Dispersion B1

While stirring 100 parts by weight of an acidic silica sol (aqueous, SNOWTEX O, manufactured by Nissan Chemical Industries, Ltd., silica content: 20%, measured pH 2.7) at room temperature, 1 part by weight of aminoguanidine sulfate was dissolved therein, and 5 parts by weight of ethanol was further added thereto, thus giving deodorizing dispersion B1. The aminoguanidine sulfate was 5 parts by weight relative to 100 parts by weight of the silica content in deodorizing dispersion B1.

### Example 203

### Preparation of Deodorizing Dispersion F1

The same procedure as for the preparation of deodorizing dispersion B1 was carried out except that 5 parts by weight of aminoguanidine sulfate and 10 parts by weight of ethanol were added, thus giving deodorizing dispersion F1. The aminoguanidine sulfate was 25 parts by weight relative to 100 parts by weight of the silica content in deodorizing dispersion F1.

### Example 204

### Storage Stability

Deodorizing dispersion B1 prepared above was stored at 30° C., and the absorbance at a wavelength of 660 nm was measured over time by a colorimeter (Novaspec II, manufactured by Pharmacia Biotech). The results are given in the Table 18.

Furthermore, the absorbance of the deodorizing dispersions and comparative samples prepared in the other Examples and Comparative Examples was also measured in the same manner, and the results are given in Table 18.

TABLE 18

| | Immedi- | | | |
|---|---|---|---|---|
| | ately after | After 1 | After 2 | After 3 |
| | preparation | week | weeks | weeks |
| Deodorizing | 0.040 | 0.106 | Gelled and | — |
| dispersion B | | | could not | |
| | | | be measured | |
| Deodorizing | 0.100 | Gelled and | — | — |
| dispersion F | | could not | | |
| | | be measured | | |
| Deodorizing | 0.038 | 0.060 | 0.082 | 0.110 |
| dispersion B1 | | | | |
| Deodorizing | 0.972 | 0.127 | 0.187 | 0.220 |
| dispersion F1 | | | | |
| Sample b | 0.152 | Gelled and | — | — |
| | | could not | | |
| | | be measured | | |
| Sample e | 0.920 | Gelled and | — | — |
| | | could not | | |
| | | be measured | | |
| Sample f | 0.042 | Gelled and | — | — |
| | | could not | | |
| | | be measured | | |

It can be seen from the above results that, with regard to the deodorizing dispersion of the present invention, adding ethanol to the dispersion medium enables the stability of the dispersion to be improved.

### Example 205

70 parts by weight of the aldehyde gas deodorant A of the present invention and 30 parts by weight of hydrated zirconium oxide as an organic acidic gas deodorant were mixed well at room temperature, thus giving deodorant composition A1 for exhaust gas containing aldehyde and acidic gases.

### Example 206

The procedure of Example 205 was carried out in the same way except that deodorant B was used instead of deodorant A, thus giving deodorant composition B1.

### Example 207

The procedure of Example 205 was carried out in the same way except that deodorant C was used instead of deodorant A, thus giving deodorant composition C1.

### Example 208

The procedure of Example 205 was carried out in the same way except that deodorant D was used instead of deodorant A, thus giving deodorant composition D1.

### Comparative Example 83

70 parts by weight of sample a and 30 parts by weight of hydrated zirconium oxide were mixed well at room temperature, thus giving sample compositional.

### Comparative Example 84

The procedure of Comparative Example 83 was carried out in the same way except that sample b was used instead of sample a, thus giving sample composition b1.

### Comparative Example 85

The procedure of Comparative Example 83 was carried out in the same way except that sample c was used instead of sample a, thus giving sample composition c1.

### Comparative Example 86

The procedure of Comparative Example 83 was carried out in the same way except that sample d was used instead of sample a, thus giving sample composition d1.

### Comparative Example 87

The procedure of Comparative Example 83 was carried out in the same way except that calcined hydrotalcite was used instead of sample a, thus giving sample composition e1.

### Example 209

### Test for Water Resistance of Deodorant Composition

The deodorizing activities of deodorant composition A1 for formaldehyde gas and formic acid were measured. Furthermore, after deodorant composition A1 was washed with purified water, the deodorizing activities for formaldehyde gas and formic acid were measured. That is, 1 g of deodorant composition A1 was placed in 100 mL of purified water at room temperature and stirred well for 1 minute. After this liquid was filtered, washing with 1000 mL of purified water and drying at 110° C. were further carried out. Deodorant composition A1 thus washed with water was subjected to measurement of deodorizing activity toward formaldehyde gas and formic acid gas. Similarly, the other deodorant compositions that had been washed with water were subjected to measurement of their deodorizing activities. Furthermore, the sample compositions prepared in the Comparative Examples were also washed with water in the same manner, and their deodorizing activities were measured.

### Example 210

### Measurement of Deodorizing Effect of Deodorant Compositions A1 to D1

Measurement of the deodorizing effect was carried out by placing in a Tedlar bag 0.02 g of deodorant composition A1 or deodorant composition A1 that had been subjected to the above water resistance test, injecting thereinto 1 L of air containing 40 ppm of formaldehyde gas and 40 ppm of formic acid gas, and allowing it to stand at room temperature for 2 hours. After 2 hours, the concentration of each gas remaining in the Tedlar bag was measured by a gas detector tube. The results are given in Table 19. Furthermore, sample compositions a1 to d1 were measured in the same manner, and the results are given in Table 19.

The same test method as above was carried out except that 1 L of air containing 40 ppm of formaldehyde and 1% of carbon dioxide and 1 L of air containing 40 ppm of formic acid and 1% of carbon dioxide were injected, and the results are given in Table 20.

TABLE 19

| | Formaldehyde | | Formic acid | |
|---|---|---|---|---|
| | Before | After | Before | After |
| | washing | washing | washing | washing |
| | with water | with water | with water | with water |
| Initial concentration | 40 | 40 | 40 | 40 |
| Deodorant | <0.1 | <0.1 | <0.5 | <0.5 |
| composition A1 | | | | |
| Deodorant | <0.1 | <0.1 | <0.5 | <0.5 |
| composition B1 | | | | |
| Deodorant | <0.1 | <0.1 | <0.5 | <0.5 |
| composition C1 | | | | |
| Deodorant | <0.1 | <0.1 | <0.5 | <0.5 |
| composition D1 | | | | |
| Sample composition | 10 | 12 | 1 | 2 |
| a1 | | | | |
| Sample composition | 10 | 12 | 1 | 2 |
| b1 | | | | |
| Sample composition | 10 | 12 | 1 | 2 |
| c1 | | | | |
| Sample composition | 10 | 12 | 1 | 2 |
| d1 | | | | |
| Sample composition | 15 | 20 | <0.5 | 1 |
| e1 | | | | |

TABLE 20

| | Formaldehyde | | Formic acid | |
|---|---|---|---|---|
| | Before | After | Before | After |
| | washing | washing | washing | washing |
| | with water | with water | with water | with water |
| Initial concentration | 40 | 40 | 40 | 40 |
| Deodorant | <0.1 | <0.1 | <0.5 | <0.5 |
| composition A1 | | | | |
| Deodorant | <0.1 | <0.1 | <0.5 | <0.5 |
| composition B1 | | | | |
| Deodorant | <0.1 | <0.1 | <0.5 | <0.5 |
| composition C1 | | | | |
| Deodorant | <0.1 | <0.1 | <0.5 | <0.5 |
| composition D1 | | | | |
| Sample composition | 15 | 20 | 3 | 5 |
| a1 | | | | |
| Sample composition | 15 | 20 | 3 | 5 |
| b1 | | | | |
| Sample composition | 15 | 20 | 3 | 5 |
| c1 | | | | |
| Sample composition | 15 | 20 | 3 | 5 |
| d1 | | | | |
| Sample composition | 25 | 30 | 6 | 10 |
| e1 | | | | |

It can be seen from the results that the deodorant composition of the present invention has high deodorizing performance for exhaust gas containing an aldehyde gas and an acidic gas compared with the Comparative Examples. Furthermore, since it has excellent water resistance, it can be used for the removal of a harmful gas such as formaldehyde or formic acid in exhaust air during fuel cell power generation.

### INDUSTRIAL APPLICABILITY

The aldehyde gas deodorant of the present invention has on its own excellent deodorizing performance toward acetaldehyde and, when used as a mixture (deodorant composition) with another deodorant showing excellent deodorizing performance toward a basic gas, a sulfurous gas, etc., it can fully exhibit acetaldehyde deodorizing performance while maintaining excellent deodorizing performance for a basic gas, a sulfurous gas, etc. The aldehyde gas deodorant or the deodorant composition of the present invention can thereby impart excellent deodorizing properties and an effect in suppressing volatilization of an aldehyde gas to fiber, paint, sheets, moldings, processed articles, etc., and they can be used as deodorizing products.

1. An aldehyde gas deodorant dispersion comprising a mixture of an aminoguanidine salt that has a pH of 1 to 7 when dissolved in purified water and an acidic silica sol, the dispersion having a pH of 1 to 7, wherein a content of the aminoguannidine salt is 0.01 to 100 parts by weight relative to 100 parts by weight of a silica (SiO₂) content of the acidic silica sol.

2. The aldehyde gas deodorizing dispersion according to claim 1, further comprising a lower alcohol.

3. The aldehyde gas deodorizing dispersion according to claim 1, further comprising a humectant.

4. The aldehyde gas deodorizing dispersion according to claim 3, wherein the humectant is a polyhydric alcohol.

5. The aldehyde gas deodorizing dispersion according to claim 1, wherein the aminoguanidine salt is selected from the group consisting of aminoguanidine sulfate, aminoguanidine hydrochloride, diaminoguanidine hydrochloride, diaminoguanidine sulfate and triaminoguanidine hydrochloride.

6. A deodorizing product produced by applying the aldehyde gas deodorant dispersion according to claim 1 to a surface of a support.

7. A deodorizing filter comprising the deodorizing product according to claim 6.

8. A deodorizing polyurethane foam comprising the deodorizing product according to 6.
